(19) United States
(12) Patent Application Publication
(10) Pub. No.: US 2025/0120948 A1
(43) Pub. Date: Apr. 17, 2025

(54) Small Molecules G(alpha)I2 Inhibitors and Combination With Anticancer Drugs and Use Thereof

(71) Applicants: Georgia Tech Research Corporation, Atlanta, GA (US); Clark Atlanta University, Atlanta, GA (US)

(72) Inventors: Shafiq Khan, Atlanta, GA (US); Adegboyega (Yomi) Oyelere, Atlanta, GA (US); Silvia Caggia, Atlanta, GA (US); Subhasish Tapadar, Atlanta, GA (US)

(21) Appl. No.: 18/907,903

(22) Filed: Oct. 7, 2024

Related U.S. Application Data
(60) Provisional application No. 63/588,038, filed on Oct. 5, 2023.

(51) Int. Cl. A61K 31/404; A61K 31/337; A61K 31/381; A61K 31/4045; A61K 31/4184

(52) U.S. Cl. CPC A61K 31/404; A61K 31/337; A61K 31/381; A61K 31/4045; A61K 31/4184

(57) ABSTRACT

An exemplary embodiment of the present disclosure provides a method for treating a subject with cancer. The method includes administering, to a subject with cancer, a chemotherapeutic drug under conditions effective to treat the subject for cancer, and administering, to the subject, a G(alpha)i2 (Gαᵢ2) inhibitor. Another embodiment of the present disclosure provides a method of reducing cell migration in a subject that is receiving a chemotherapeutic drug to inhibit cancer cells. The method includes administering a Gαᵢ2 inhibitor.

### CROSS-REFERENCE TO RELATED APPLICATIONS

This application claims the benefit of U.S. Provisional Application Ser. No. 63/588,038, filed on Oct. 5, 2023, which is incorporated herein by reference in its entirety as if fully set forth below.

### FIELD OF THE DISCLOSURE

The various embodiments of the present disclosure relate generally to small molecules G(alpha)I2 inhibitors and combination with anticancer drugs and use thereof.

### BACKGROUND

Prostate cancer is the second most diagnosed and the second leading cause of cancer deaths among American men. According to American Cancer Society, 288,300 men will be diagnosed, and 34,700 men will die of prostate cancer in the U.S. in 2023. One of the common treatment approaches for AR-positive prostate cancers is androgen deprivation therapy (ADT), based on either inhibition of biosynthesis and/or action of androgens. However, these treatments ultimately lead to the development of castration-resistant prostate cancer (CRPC) in which the cancer cells develop resistance to these treatments. For CRPC patients, docetaxel has been used as a standard first-line chemotherapy regimen. Additionally, combination of ADT and docetaxel has also been introduced to treat hormone sensitive prostate cancers, a therapeutic regimen that has increased the patient's overall survival. Depending on the cancer type and the stage of the disease, surgery followed by chemotherapy is the most common strategy utilized to treat cancer patients that could prove effective and curative. However, the development of distant metastases is quite common in cancer patients despite achieving complete control of the primary tumor. Traditionally, it has been assumed that these metastases existed as undetectable cancer foci, which were already present at the time of initial treatments.

However, recent studies have shown that chemotherapy itself can also increase the capability of cancer cells to escape from death and migrate to distant sites to form metastases. A class of chemotherapeutic agents, which have shown benefits in the management of hematological malignancies, are histone deacetylase inhibitors (HDACi). The potential of HDACi as therapeutic agents for solid tumors, including prostate cancer, is also being investigated in several pre-clinical and clinical studies. The inventors have previously developed very potent HDAC inhibitors conjugated with androgen receptor antagonists which were shown to be highly cytotoxic for prostate cancer cells in vitro and in vivo. However, recent studies indicate that HDACi could elicit a double-edge sword effect: while these inhibitors have potent effects on inducing cell death and inhibition of tumor growth, they can increase the risk of metastatic disease by inducing cancer cell invasion and migration. The cellular and molecular mechanisms involved in these divergent effects are not well defined. However, these studies suggest that as a response to the cytotoxic effects of chemotherapy, the tumor cells may not only develop drug resistance but the altered tumor microenvironment may also induce migratory and invasive behavior in target cells, which may lead to increased metastasis. Such effects of chemotherapy on migratory and invasive behavior have not been well investigated in prostate cancers.

The present invention is directed to overcoming these and other deficiencies in the art.

### BRIEF SUMMARY

An exemplary embodiment of the present disclosure provides a method for treating a subject with cancer. The method includes administering, to a subject with cancer, a chemotherapeutic drug under conditions effective to treat the subject for cancer, and administering, to the subject, a G(alpha)i2 (Gαᵢ2) inhibitor.

Another embodiment of the present disclosure provides a method of reducing cell migration in a subject that is receiving a chemotherapeutic drug to inhibit cancer cells. The method includes administering a Gαᵢ2 inhibitor.

In any of the embodiments disclosed herein, the Gαᵢ2 inhibitor can be a compound of Formula I having the following structure:

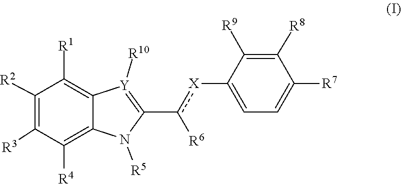

where [symbol] is a single or double bond, R¹, R², and R³ are each independently H, —OH, —NH₂, or halogen, R⁴ is H, alkyl, halo-alkyl, aryl, pyridyl, wherein the aryl and pyridyl groups are optionally substituted at the ortho, meta, and para positions, R⁵ is H, methyl, or ethyl, R⁶ is H methyl, or ethyl, R⁷ is —OH, -OMe, -SMe, -SOMe, or —SO₂Me, R⁸ and R⁹ are each independently H, or —C(O)NH₂OH, R¹⁰ is H, —OH, or halogen, X is N or O, and Y is C or N.

In any of the embodiments disclosed herein, the Gαᵢ2 inhibitor can be selected from the group consisting of

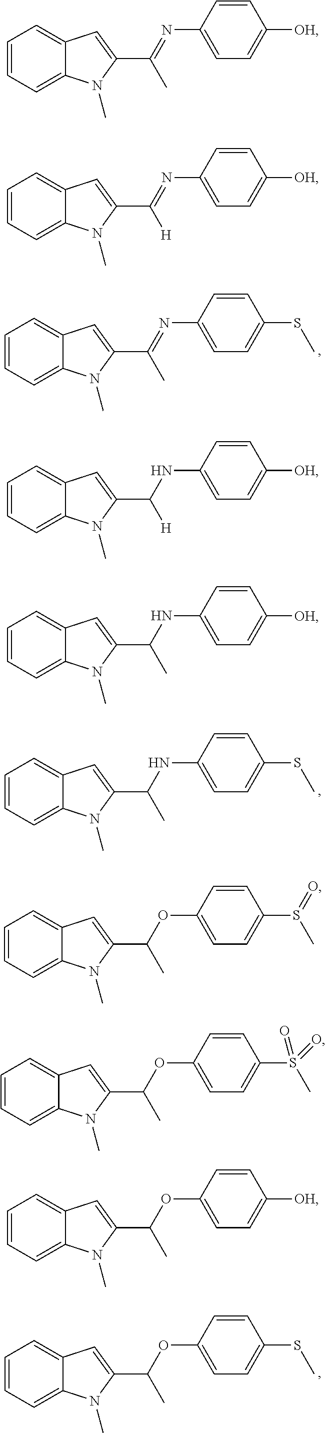

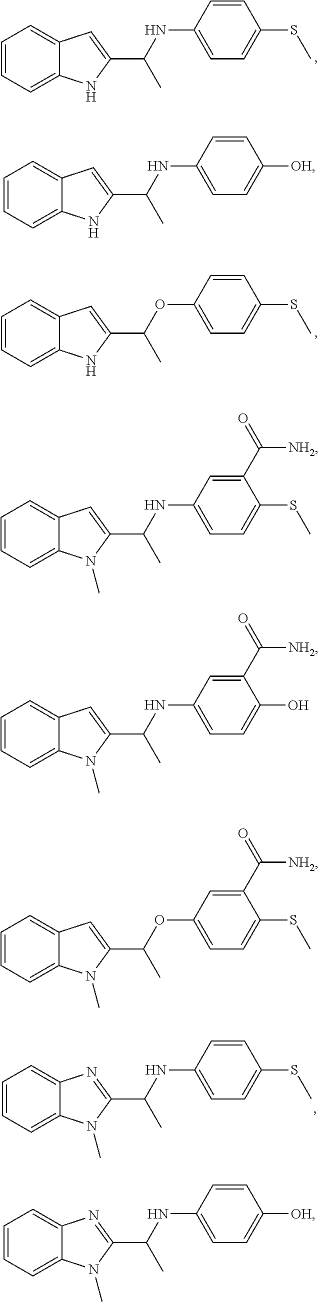

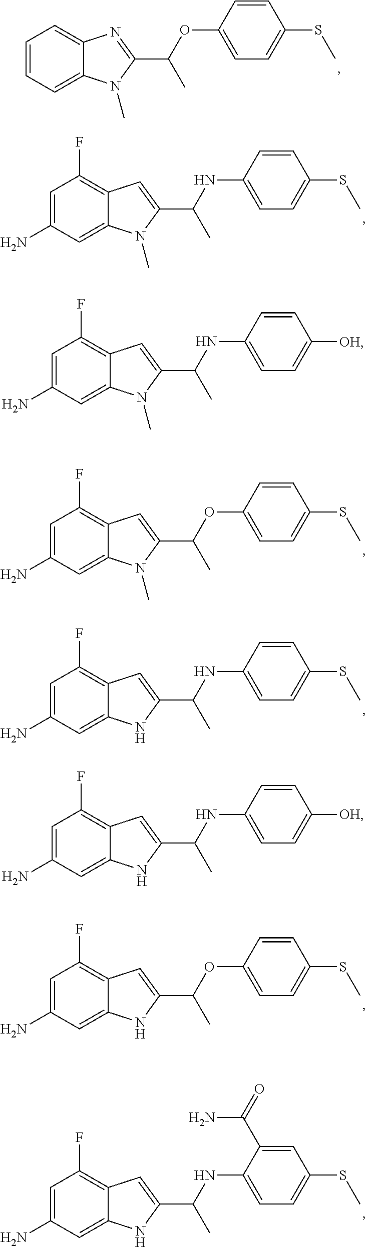

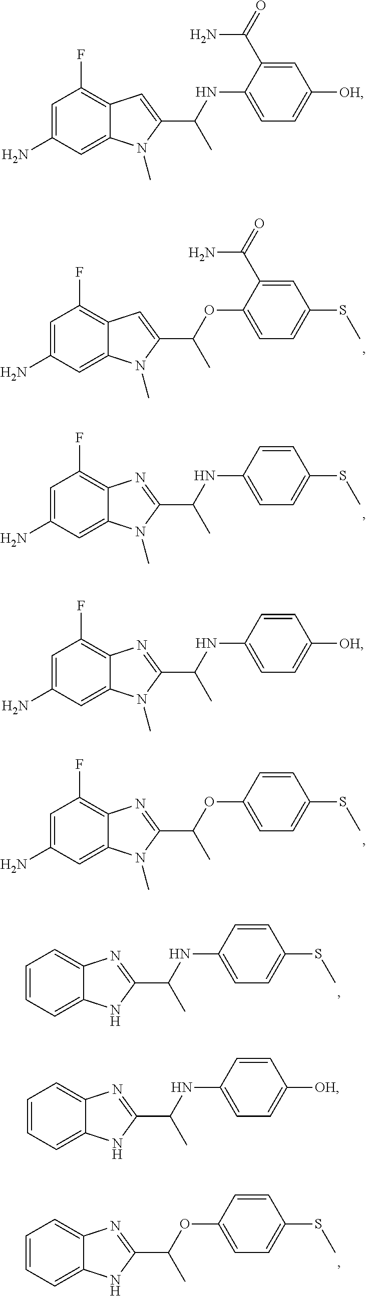

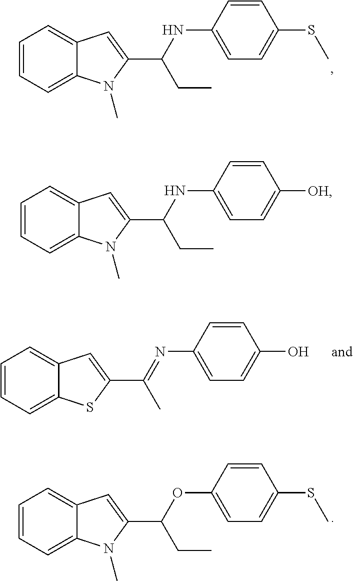 [SMILES: CCC(Nc1ccc(SC)cc1)c1cc2ccccc2n1C ; CCC(Nc1ccc(O)cc1)c1cc2ccccc2n1C ; C/C(=N\c1ccc(O)cc1)c1cc2ccccc2s1 ; CCC(Oc1ccc(SC)cc1)c1cc2ccccc2n1C]

In some embodiments, the Gαᵢ2 inhibitor is

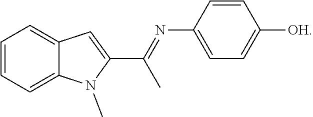 [SMILES: C/C(=N\c1ccc(O)cc1)c1cc2ccccc2n1C]

In some embodiments, the Gαᵢ2 inhibitor is

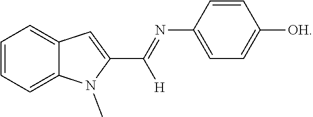 [SMILES: Cn1c(/C=N/c2ccc(O)cc2)cc2ccccc21]

In some embodiments, the Gαᵢ2 inhibitor is

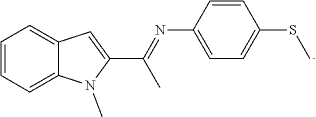 [SMILES: CSc1ccc(/N=C(\C)c2cc3ccccc3n2C)cc1]

In some embodiments, the Gαᵢ2 inhibitor is

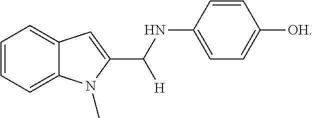 [SMILES: Cn1c(CNc2ccc(O)cc2)cc2ccccc21]

In some embodiments, the Gαᵢ2 inhibitor is

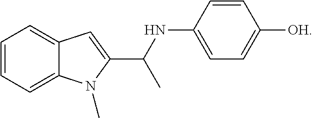 [SMILES: CC(Nc1ccc(O)cc1)c1cc2ccccc2n1C]

In some embodiments, the Gαᵢ2 inhibitor is

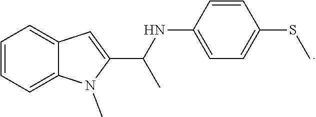 [SMILES: CSc1ccc(NC(C)c2cc3ccccc3n2C)cc1]

In some embodiments, the Gαᵢ2 inhibitor is

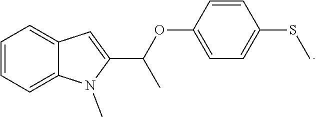 [SMILES: CSc1ccc(OC(C)c2cc3ccccc3n2C)cc1]

In some embodiments, the Gαᵢ2 inhibitor is

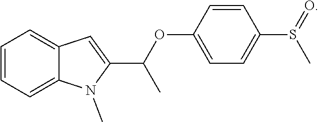 [SMILES: CC(Oc1ccc(S(C)=O)cc1)c1cc2ccccc2n1C]

In some embodiments, the G(alpha)ᵢ2 inhibitor is

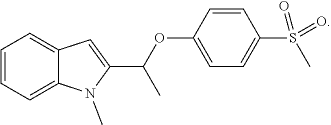 [SMILES: CC(Oc1ccc(S(C)(=O)=O)cc1)c1cc2ccccc2n1C]

In any of the embodiments disclosed herein, the chemotherapeutic drug can induce cell migration if the subject does not receive the Gαᵢ2 inhibitor.

In any of the embodiments disclosed herein, the Gαᵢ2 inhibitor can inhibit cancer cell migration that is induced by the chemotherapeutic drug.

In any of the embodiments disclosed herein, the cancer can be selected from the group consisting of prostate cancer, breast cancer, and ovarian cancer.

In any of the embodiments disclosed herein, the cancer can be prostate cancer.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-1B show that second generation compounds differently modulated cell migration in PC3 prostate cancer cell lines, in accordance with some embodiments of the present disclosure. PC3 cells were incubated with (+) or without (−) compounds 3-7, 10, 12-13 and 15, at several concentrations, and then subjected to transwell migration assay. Epidermal growth factor (EGF) (10 ng/ml) was used as positive control. Results are expressed as migration index. Each bar represents mean±(SEM) (n=3). The asterisks on each bar represent significant differences (P<0.05) among various treatment groups, compared to the controls.

FIGS. 2A-2B provide Dock poses of enantiomers of 6 (6-R and 6-S) and 15 (15-R and 15-S) at Gαᵢ active site, in accordance with some embodiments of the present disclosure. FIG. 2A shows that 6-R is accommodated at the Gαᵢ active site with a slightly enhanced binding affinity relative to 6-S(8.6 kcal/mol vs 8.1 kcal/mol). FIG. 2B shows that 15-R and 15-S bind to Gia with the same binding affinity (8.0 kcal/mol), which is relatively weaker to that of 6-R.

FIGS. 3A-3B provide data for HDACi induced cell migration in LNCaP prostate cancer cell lines, in accordance with some embodiments of the present disclosure. LNCaP cells were incubated with (+) or without (−) SAHA (FIG. 3A) or SBI-I-19 (FIG. 3B), at several concentrations, and then subjected to transwell migration assay. EGF (10 ng/ml) was used as positive control. Results are expressed as migration index. Each bar represents mean±SEM (n=3). The asterisks on each bar represent significant differences (P<0.05) among various treatment groups, compared to the controls. Chemical structures of SAHA and SBI-I-19 are depicted with respective bar diagrams.

FIGS. 4A-4B provide data for anti-androgens induced cell migration in LNCaP prostate cancer cell lines, in accordance with some embodiments of the present disclosure. LNCaP cells were incubated with (+) or without (−) enzalutamide (FIG. 4A) or bicalutamide (FIG. 4B), at several concentrations, and then subjected to transwell migration assays. EGF (10 ng/ml) was used as positive control. Results are expressed as migration index. Each bar represents mean±SEM (n=3). The asterisks on each bar represent significant differences (P<0.05) among various treatment groups, compared to the controls. Chemical structures of enzalutamide and bicalutamide are depicted with respective bar diagrams.

FIG. 5 provides the effects of compounds 3-7, 10, 12, 13 and 15 on the viability of LNCaP, MDA-MD-231, MCF-7 and Vero cells, in accordance with some embodiments of the present disclosure. The cells are incubated with the drugs for 72 hours and viability evaluated with MTS. NI=IC50 greater than 100 μM, n=3

FIGS. 6A-6D provide dose-response curves showing the effects of compounds 3-7, 10, 12, 13 and 15 on the viability of LNCaP (FIG. 6A), MDA-MB-231 (FIG. 6B), MCF-7 (FIG. 6C) and Vero (FIG. 6D) cells, in accordance with some embodiments of the present disclosure.

FIG. 7 shows cell viability and proliferation of LNCaP cells after treatments with chemotherapeutic drugs, in accordance with some embodiments of the present disclosure.

FIGS. 8A-8C provide data for docetaxel, SAHA and anti-androgen effects on cell migration in PC3 and DU145 prostate cancer cell lines, in accordance with some embodiments of the present disclosure. PC3 (FIGS. 8A-8B) and DU145 (FIG. 8C) cells were incubated with (+) or without (−) different agents, and then subjected to transwell migration assays. EGF (10 ng/ml) was used as positive control. Results are expressed as migration index. Each bar represents mean±SEM (n=3). The asterisks on each bar represent significant differences (P<0.05) among various treatment groups, compared to the controls. The chemical structure of docetaxel is depicted with respective bar diagrams.

FIG. 9 shows that anti-androgens induced an increase in the levels of Gαᵢ2 protein in LNCaP cell lines, in accordance with some embodiments of the present disclosure. LNCaP cells were incubated for 24 hours with enzalutamide (2.5 μm), bicalutamide (2.5 μM), SAHA (4 μM) and SBI-I-19 (2 μM) and then subjected to western blot analysis, to evaluate the expression of Gαᵢ2 protein. DMSO was used as control. Results are expressed as ratio between Gαᵢ2 band densitometry/α-tubulin band densitometry. Each bar represents mean±SEM (n=3). The asterisks on each bar represent significant differences (P<0.05) against the control (DMSO).

FIGS. 10A-10E show that combination therapies with chemotherapeutic drugs and Gαᵢ2 inhibitor decreased prostate cancer cell migration, in accordance with some embodiments of the present disclosure. Prostate cancer cells were incubated with (+) or without (−) Docetaxel (FIG. 10A, PC3), enzalutamide (FIG. 10B, LNCaP), bicalutamide (FIG. 10C, LNCaP), SAHA (FIG. 10D, LNCaP) and SBI-I-19 (FIG. 10E, LNCaP) at appropriate concentrations, with (+) or without (−) compound 14 (25 μM), and then subjected to transwell migration assay in the presence or absence of EGF (10 ng/ml). Results are expressed as migration index. The chemical structure of compound 14 is depicted in the left top corner. Each bar represents mean±SEM (n=3). The asterisks on each bar represent significant differences (P<0.05) among various treatment groups, compared to the controls.

FIGS. 11A-11B show that combination therapies with SAHA or docetaxel and the second generation Gαᵢ2 inhibitors decreased PC3 cell migration, in accordance with some embodiments of the present disclosure. PC3 cells were incubated with (+) or without (−) SAHA (FIG. 11A) or docetaxel (FIG. 11B) at appropriate concentrations, with (+) or without (−) compound 14 (20 μM), and the second generation of Gαᵢ2 inhibitors (compounds 6, 10, 7, at 20 μM) and then subjected to transwell migration assay in the presence or absence of EGF (10 ng/ml). Results are expressed as migration index. Each bar represents mean±SEM (n=3). The asterisks on each bar represent significant differences (P<0.05) among various treatment groups, compared to the controls.

FIGS. 12A-12C show that new small molecule inhibitors of Gαᵢ2 protein block the stimulatory effects of EGF on PC3 cell migration, in accordance with some embodiments of the present disclosure. The compounds shown in FIGS. 12A-12C correspond to the compounds of Scheme 1 as follows: AO-3-17 is compound 3, AO-3-25 is compound 5, AO-3-28 is compound 6, and AO-3-36 is compound 10.

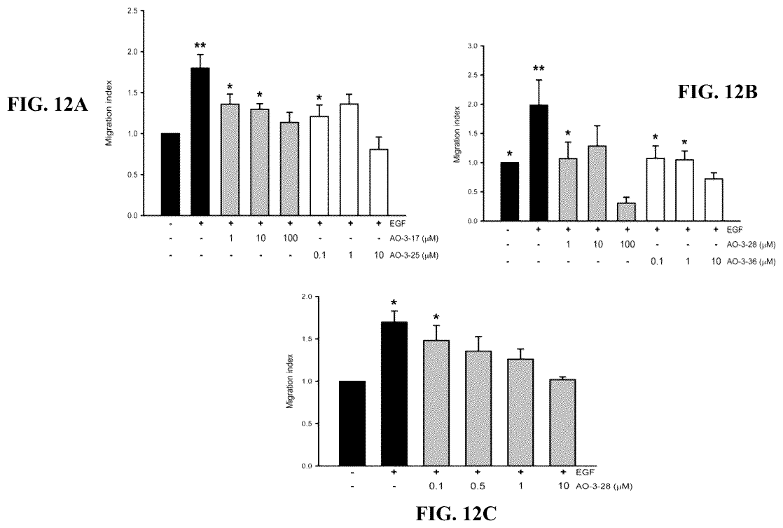

FIG. 13 shows that compounds 14 and 6, inhibitors of Gαᵢ2 protein, block the stimulatory effects of Docetaxel on PC3 cell migration, in accordance with some embodiments of the present disclosure.

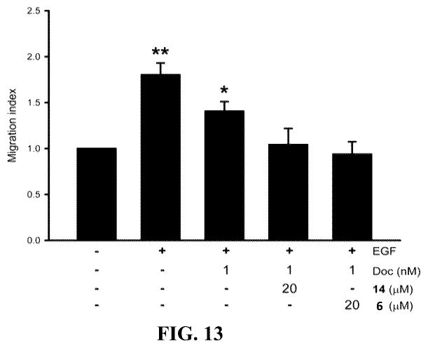

FIG. 14 shows that compound 7, inhibitor of Gαᵢ2 protein, blocks the stimulatory effects of EFG on PC3 cell migration, in accordance with some embodiments of the present disclosure. The compounds shown in FIG. 14 correspond to the compounds of Scheme 1 as follows: AO-3-54 is compound 7, AO-3-56A is compound 12, AO-3-53 is compound 4, and AO-3-56B is compound 13.

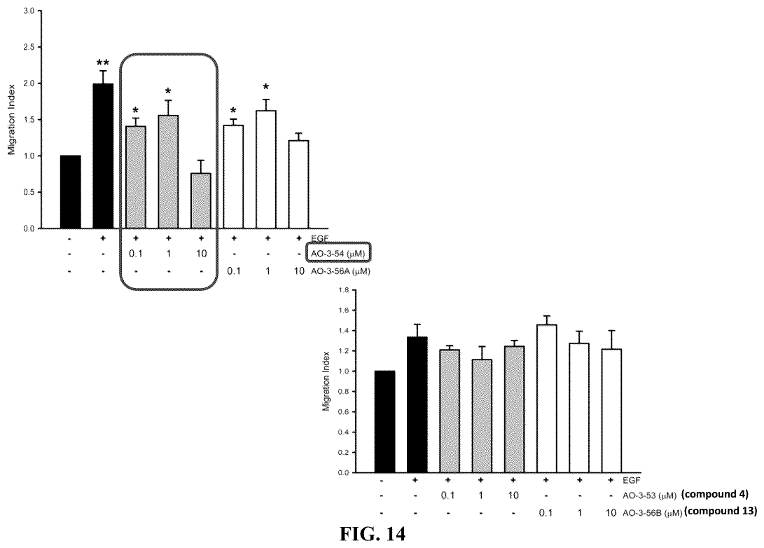

FIG. 15 provides PC3 MTS assay data showing that new small molecule inhibitors of Gαᵢ2 protein are not cytotoxic at the concentrations used to perform in vitro cell migration assays in PC3 cells, in accordance with some embodiments of the present disclosure.

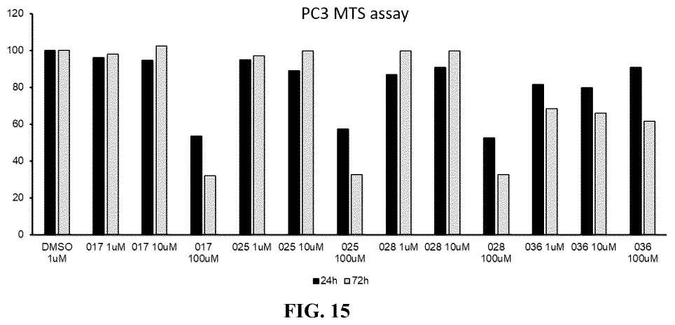

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

The present disclosure generally relates to small molecule inhibitors of Gαᵢ2 and methods of their use in combination therapies with anticancer drugs to inhibit cancer cell migration and metastasis. The small molecules can work by inhibiting through a mechanism that includes but is not limited to keeping the Gαᵢ2 protein in its inactive GDP-bound state, resulting in the inhibition of EGF-, oxytocin- and chemotherapy-stimulated migration of several cancer cells including but not limited to prostate, breast, and ovarian cancer cells. Some embodiments of the present disclosure describe the method for making the Gαᵢ2 inhibitors, the combination therapy of the Gαᵢ2 inhibitors with anticancer drugs to mitigate the intrinsic and chemotherapy-induced cancer cell migration and metastasis.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

As used in the specification and the appended claims, the singular forms “a,” “an” and “the” include plural referents unless the context clearly dictates otherwise.

The term “alkyl” means an aliphatic hydrocarbon group which may be straight or branched. When not otherwise restricted, the term refers to an alkyl of 20 or fewer carbons. Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, 3-pentyl, and the like.

The term “aryl” means an aromatic monocyclic or multi-cyclic (polycyclic) ring system of 6 to about 19 carbon atoms, or of 6 to about 10 carbon atoms. The ring system of the aryl group may be optionally substituted. Representative aryl groups include, but are not limited to, groups such as phenyl, naphthyl, azulenyl, phenanthrenyl, anthracenyl, fluorenyl, pyrenyl, triphenylenyl, chrysenyl, and naphthacenyl.

The term “halogen” means fluoro, chloro, bromo, or iodo.

The term “optionally substituted” is used to indicate that a group may have a substituent at each substitutable atom of the group (including more than one substituent on a single atom), provided that the designated atom's normal valency is not exceeded, and the identity of each substituent is independent of the others. The referenced group may be substituted with one or more additional group(s), including various known protecting groups. The protecting groups that may form the protected derivatives of the above substituents are known to those of skill in the art and may be found in references such as Greene and Wuts. Unless otherwise specified, any of the substituents described herein can be substituted or unsubstituted. For example, “alkyl” can include, e.g., propyl or substituted propyl (e.g., propyl bromide).

Also, in describing the preferred exemplary embodiments, terminology will be resorted to for the sake of clarity. It is intended that each term contemplates its broadest meaning as understood by those skilled in the art and includes all technical equivalents that operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Chemotherapy is normally the first line of treatment in cancer patients. Depending on the cancer types and the stages of the disease, this strategy can be effective and curative. However, the development of distant metastases is quite common in cancer patients. Recent studies have shown that chemotherapy can induce the cancer cells to escape from death and migrate to distant sites to form metastasis in several cancer types. However, not much is known about the effects of chemotherapy on metastasis in prostate cancer patients. The inventors evaluated the effects of chemotherapeutic drugs, such as taxanes (docetaxel), anti-androgens (enzalutamide and bicalutamide) and histone deacetylase (HDAC) inhibitors (SAHA and SBI-I-19), on cell migration in LNCaP, DU145 and PC3 prostate cancer cells. All treatments were observed to induce cell migration in LNCaP cells. Similarly, treatment with enzalutamide and HDACi induced DU145 cell migration. Docetaxel and SAHA caused a significant increase in cell migration in androgen receptor (AR)-negative PC3 cells. Previously, the inventors have shown that heterotrimeric G-protein subunit alphai2 (Gαᵢ2) is essential for cell migration and invasion in prostate cancer cells and small molecule inhibitors targeting Gαᵢ2 inhibit the migratory and invasive behavior of several cancer cell types. The sequence of Gαᵢ2 is provided in the sequence listing for the inventors' previous patent, U.S. Pat. No. 12,084,418, which is hereby incorporated by reference in its entirety. In the present study, anti-androgens significantly upregulated the levels of Gαᵢ2 protein in LNCaP cells and simultaneous treatments with Gαᵢ2 inhibitors blocked prostate cancer cell migration induced by all chemotherapeutic agents. These results show for the first time that chemotherapy may induce prostate cancer cell migration and invasion, and a combination treatment with Gαᵢ2 inhibitors and chemotherapy could blunt the capability of cancer cells to migrate and form metastasis.

The inventors have previously shown that Gαᵢ2 protein, a subunit of the heterotrimeric G-proteins, plays an essential role during prostate cancer cell migration and invasion (Zhong et al., “The Essential Role of Gialpha2 in Prostate Cancer Cell Migration,” Mol Cancer Res, 10:1380-1388 (2012); Caggia et al., “Novel Role of Gialpha2 in Cell Migration: Downstream of PI3-kinase-AKT and Rac1 in Prostate Cancer Cells,” J Cell Physiol, 234:802-815 (2018)). Moreover, inventors have synthesized a cohort of small molecule inhibitors, which block the activation of the Gαᵢ2 protein, leading to inhibition of cell migration and invasion in several cancer cell types (Caggia et al., “Small Molecule Inhibitors Targeting Galphai2 Protein Attenuate Migration of Cancer Cells,” Cancers (Basel), 12(6):1631 (2020)). The current study was carried out to optimize the first generation Gαᵢ2 inhibitors, determine the possible effects of representative chemotherapeutic drugs (HDACi, anti-androgens and taxanes) on cell migration in prostate cancer cells and whether these effects can be blocked by the treatment with these Gαᵢ2 inhibitors.

An exemplary embodiment of the present disclosure provides a method for treating a subject with cancer. The method includes administering, to a subject with cancer, a chemotherapeutic drug under conditions effective to treat the subject for cancer, and administering, to the subject, a Gαᵢ2 inhibitor.

Another embodiment of the present disclosure provides a method of reducing cell migration in a subject that is receiving a chemotherapeutic drug to inhibit cancer cells. The method includes administering a Gαᵢ2 inhibitor.

“Treating cancer” as used herein, specifically refers to administering therapeutic agents to a subject diagnosed with cancer, i.e., having established cancer in the subject, to inhibit the further growth or spread of the malignant cells in the cancerous tissue, and/or to cause the death of the malignant cells. In particular, prostate cancer, breast cancer, ovarian cancer, renal cancer, CNS cancer, melanoma, lung cancer, and bladder cancer are amenable to the treatment in accordance with the method of the present invention. Treating cancer also encompasses treating a subject having premalignant conditions to stop the progression of, or cause regression of, the premalignant conditions.

In practicing the method of treating a cancer in a subject of the present disclosure, the administering step is carried out by administering a chemotherapeutic drug and a Gαᵢ2 inhibitor (i.e., a compound of Formula I), or a pharmaceutically acceptable salt thereof, orally, intradermally, intramuscularly, intraperitoneally, intravenously, subcutaneously, or intranasally. The Gαᵢ2 inhibitor of the present invention may be administered alone or with suitable pharmaceutical carriers and optionally, other therapeutic ingredients or excipients, and can be in solid or liquid form, such as tablets, capsules, powders, solutions, suspensions, or emulsions.

The magnitude of the Gαᵢ2 inhibitor, or a pharmaceutically acceptable salt thereof, will vary with the nature and severity of the condition to be treated and the route of administration. The dose, and perhaps the dose frequency, will also vary according to the age, body weight, and response of the individual subject. The total daily dose may be administered in single or divided doses.

The term “pharmaceutically acceptable salt thereof” refers to salts prepared from pharmaceutically acceptable, non-toxic acids including inorganic acids and organic acids, such as, for example, acetic acid, benzenesulfonic (besylate) acid, benzoic acid, camphorsulfonic acid, citric acid, ethenesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, and p-toluenesulfonic acid.

The compositions of the present disclosure may include a pharmaceutically acceptable carrier. The carrier may take a wide variety of forms, depending on its desired for administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets. Preferred solid oral preparations are tablets or capsules, because of their ease of administration. If desired, tablets may be coated by a standard aqueous or nonaqueous technique. Oral and parenteral sustained release dosage forms may also be used.

The pharmaceutical compositions may also be administered in injectable dosages by solution or suspension of these materials in a physiologically acceptable diluent with a pharmaceutical carrier. Such carriers include sterile liquids, such as water and oils, with or without the addition of a surfactants, adjuvants, excipients, or stabilizers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

As used herein, the term “subject” or “patient” refers to mammals and includes, without limitation, human and veterinary animals. Preferred subjects for treating cancer in accordance with the methods of the present invention include, without limitation, any mammal, preferably a human.

In some embodiments, the Gαᵢ2 inhibitor can be a compound of Formula I having the following structure:

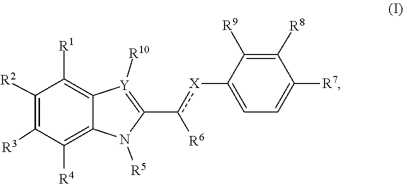

where [symbol] is a single or double bond, R¹, R², and R³ are each independently H, —OH, —NH₂, or halogen, R⁴ is H, alkyl, halo-alkyl, aryl, pyridyl, wherein the aryl and pyridyl groups are optionally substituted at the ortho, meta, and para positions, R⁵ is H, methyl, or ethyl, R⁶ is H methyl, or ethyl, R⁷ is —OH, -OMe, -SMe, -SOMe, or —SO₂Me, R⁸ and R⁹ are each independently H, or —C(O)NH₂OH, R¹⁰ is H, —OH, or halogen, X is N or O, and Y is C or N.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds should be interpreted as within the scope of the disclosure. Accordingly, for example, the present disclosure should be construed as disclosing treatment with each enantiomer separately and treating with a mixture of enantiomers.

In some embodiments the Gαᵢ2 inhibitor is selected from the group consisting of

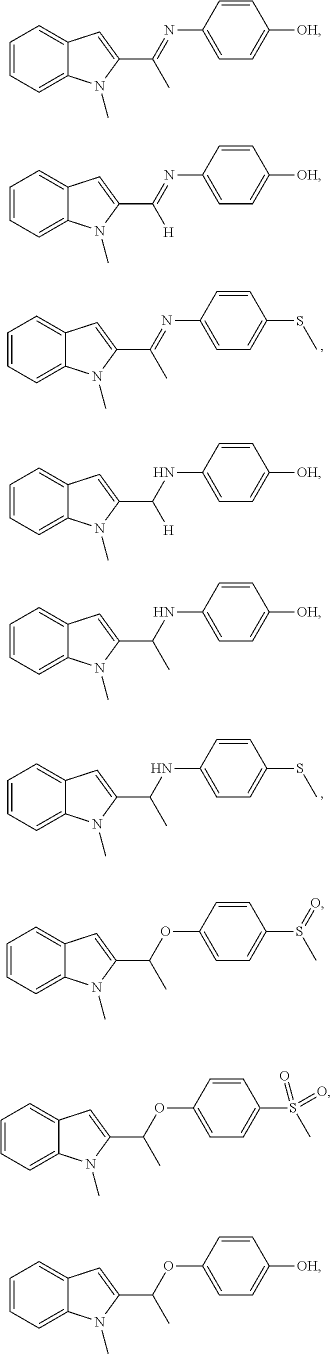

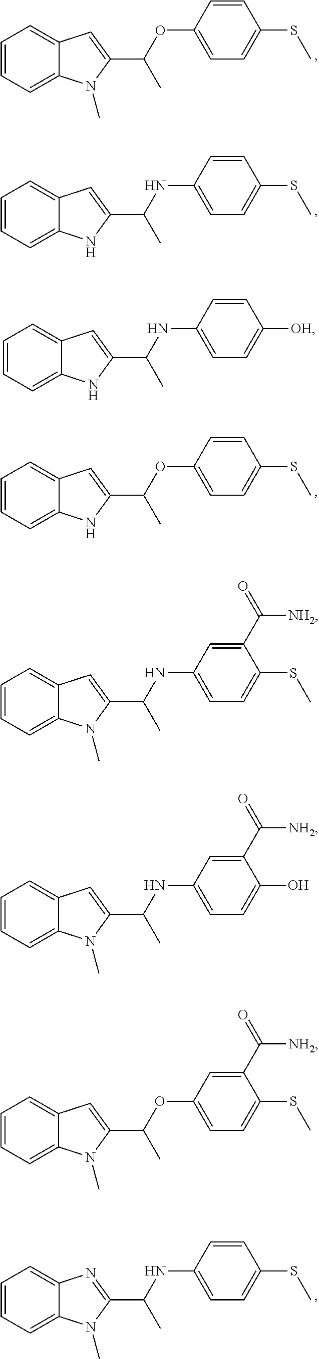

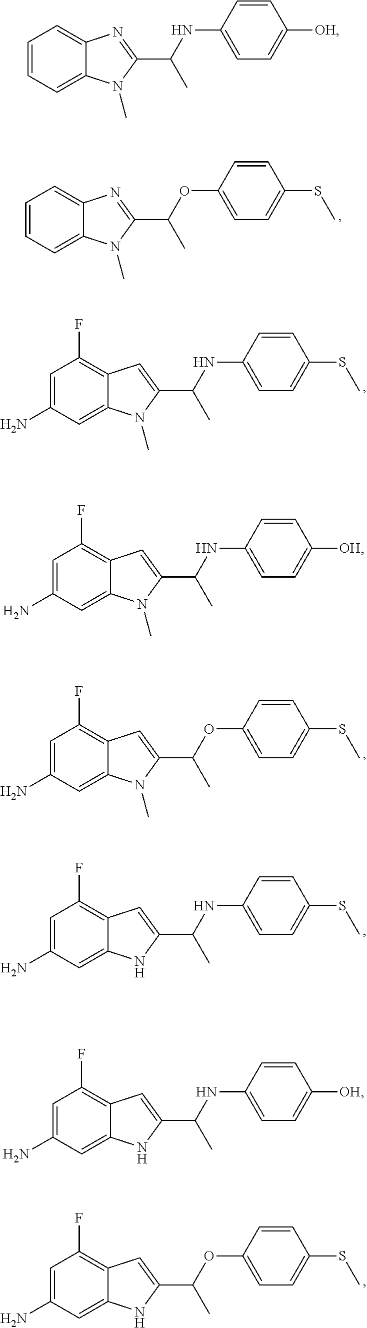

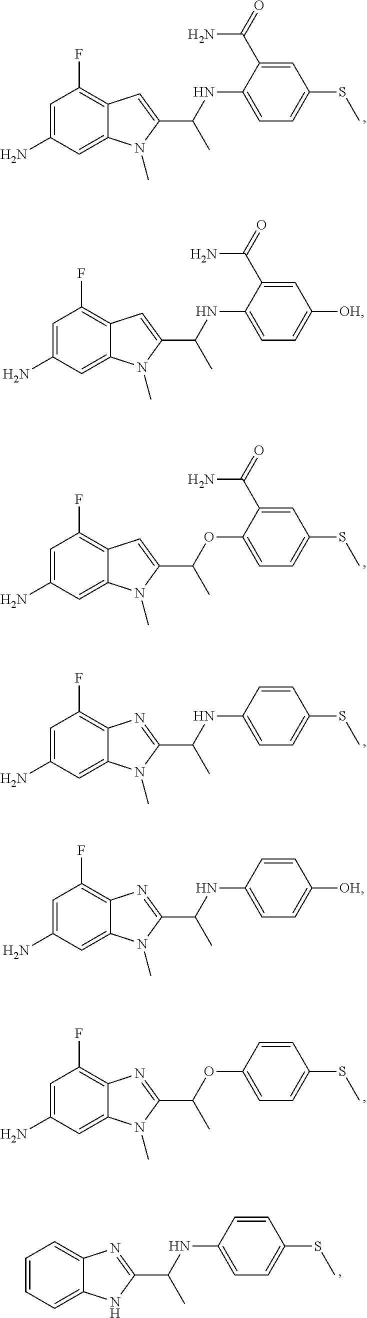

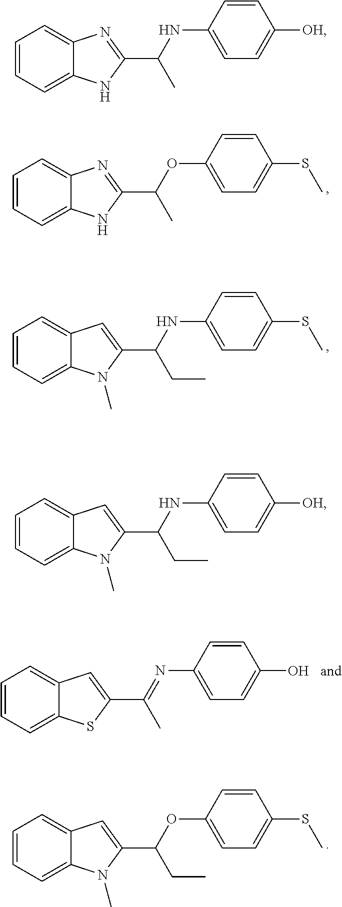

In some embodiments, the chemotherapeutic drug induces cell migration if the subject does not receive the Gαᵢ2 inhibitor.

In some embodiments, the Gαᵢ2 inhibitor inhibits cancer cell migration that is induced by the chemotherapeutic drug.

As used herein, the term “chemotherapeutic drug” refers to any agent used to treat cancer by killing or slowing the growth of cancerous cells. The method of the present disclosure can be carried out with any chemotherapeutic drug. Exemplary chemotherapeutic drugs can include docetaxel, SAHA, SBI-I-19, enzalutamide, and bicalutamide.

In any embodiments of the present disclosure, the cancer can be prostate cancer, breast cancer, or ovarian cancer. In a preferred embodiment, the cancer is prostate cancer.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

### EXAMPLES

The following Examples are presented to illustrate various aspects of the present application, but are not intended to limit the scope of the claimed application.

### Example 1—Materials and Methods for Examples 2-9

### Chemicals and Reagents

Reagents and solvents for synthesis were purchased from Sigma-Aldrich (St. Louis, MO, USA), VWR International, (Radnor, PA, USA), Combi-blocks and Ambeed and were used as received. Analtech silica gel plates (60 F254) were used for analytical TLC, and plates were visualized using UV light, anisaldehyde, and/or iodine stains. Analtech preparative TLC plates (UV254, 2000 m) and silica gel (200-400 mesh) were used for purification by prep-TLC and column chromatography, respectively. Compounds were characterized using NMR spectra obtained on a Varian-Gemini 400 MHz and Bruker Ascend™ 500 and 700 MHz magnetic resonance spectrometer. ¹H NMR spectra were recorded in parts per million (ppm) relative to the residual peaks of CHCl₃ (7.24 ppm) in CDCl₃ or DMSO-d5 (2.49 ppm) in DMSO-d6. ¹³C NMR spectra were recorded relative to the CDCl₃ triplet peak (77.0 ppm), or the DMSO-d6 septet (39.7 ppm) and were recorded with complete heterodecoupling.

Multiplicities are described using the abbreviation s, singlet; d, doublet, t, triplet; q, quartet; m, multiplet; and app, apparent. MestReNova (version 11.0) was used to process the “fid” files. Low- and high-resolution mass spectra were obtained at the Georgia Institute of Technology mass spectrometry facility (Atlanta, GA, USA).

### Cell Culture and Reagents

LNCaP human AR+(ATCC® CRL-1740™), DU145 (ATCC® HTB-81™) and PC3 human AR− (ATCC® CRL-1435™) prostate cancer cell lines were obtained from American Type Culture Collection (ATCC) (Rockville, MD). They were maintained as previously described (Caggia et al., “Novel Role of Gialpha2 in Cell Migration: Downstream of PI3-kinase-AKT and Rac1 in Prostate Cancer Cells,” J Cell Physiol, 234:802-815 (2018); Caggia et al., “Small Molecule Inhibitors Targeting Galphai2 Protein Attenuate Migration of Cancer Cells,” Cancers (Basel), 12(6):1631 (2020)), and they were tested periodically for mycoplasma contamination, using PlasmoTest™—Mycoplasma Detection Kit (InvivoGen, San Diego, CA). Anti-α-tubulin and bovine serum albumin (BSA) were obtained from Sigma-Aldrich (St. Louis, MO, USA). Rat tail collagen and transwell inserts were obtained from BD Biosciences (San Jose, CA, USA). DAPI (4′,6-Diamidino-2-Phenylindole) was purchased from Invitrogen through Thermo Fisher Scientific (Eugene, OR, USA). Rabbit polyclonal anti-Gαᵢ2 antibody (ab 157204) was purchased from Abcam (Cambridge, MA, USA). Epidermal growth factor (EGF) was obtained from Life Technologies (Grand Island, NY, USA). The anti-rabbit and anti-mouse immunoglobulins coupled with horseradish peroxidase (IgG-HRP), were obtained from Promega (Madison, WI, USA). Cell culture reagents were purchased from Corning Life Sciences (Tewksbury, VA, USA).

### Treatments

Docetaxel, a taxoid anticancer agent, was purchased from Millipore-Sigma, and dissolved in DMSO at a starting concentration of 100 μM. SAHA, a commercially available FDA-approved HDACi, was purchased from Sigma-Aldrich (St. Louis, MO), and dissolved in DMSO at a starting concentration of 100 mM. SBI-I-19, an HDAC inhibitor conjugated with anti-androgen (Gryder et al., “Selectively Targeting Prostate Cancer with Antiandrogen Equipped Histone Deacetylase Inhibitors,” ACS Chem Biol, 8:2550-2560 (2013)), was dissolved in DMSO at a starting concentration of 50 mM. The anti-androgen drugs enzalutamide and bicalutamide were purchased from Selleckchem (Houston, TX) and Sigma-Aldrich, respectively. Anti-androgens were prepared at a stock concentration of 100 mM (enzalutamide) and 10 mM (bicalutamide) in DMSO. The Gαᵢ2 inhibitor (compound 14) was diluted in DMSO, at a stock concentration of 100 mM, as previously described (Caggia et al., “Small Molecule Inhibitors Targeting Galphai2 Protein Attenuate Migration of Cancer Cells,” Cancers (Basel), 12(6):1631 (2020)). All drugs used in the study were then diluted in the culture media to the final concentrations used in various assays (Caggia et al., “Novel Role of Gialpha2 in Cell Migration: Downstream of PI3-kinase-AKT and Rac1 in Prostate Cancer Cells,” J Cell Physiol, 234:802-815 (2018); Caggia et al., “Small Molecule Inhibitors Targeting Galphai2 Protein Attenuate Migration of Cancer Cells,” Cancers (Basel), 12(6):1631 (2020)). To determine the effects of specific treatments on the levels of Gαᵢ2 protein, LNCaP and PC3 cells (10⁶ cells) were cultured overnight in 10 cm² culture dishes. The cells were then treated with anti-androgens and other agents for 24 hours and the total cell lysates were collected for western blot analyses.

### Cell Migration Assays

In vitro transwell migration assays were conducted as described previously (Caggia et al., “Novel Role of Gialpha2 in Cell Migration: Downstream of PI3-kinase-AKT and RacI in Prostate Cancer Cells,” J Cell Physiol, 234:802-815 (2018); Caggia et al., “Small Molecule Inhibitors Targeting Galphai2 Protein Attenuate Migration of Cancer Cells,” Cancers (Basel), 12(6):1631 (2020)). Briefly, the outside of the transwell inserts (BD Biosciences, San Jose, CA) were coated with 50 μl of rat tail collagen (50 g/ml; BD Biosciences, San Jose, CA) overnight at 4° C. The next day, 50 μl of the aforementioned rat tail collagen were added for 1 hour at room temperature into the transwell inserts, to coat the inside of the membranes. DU145, PC3 and LNCaP cells were trypsinized and suspended at the appropriate density (3×10⁵ cells/insert for DU145 and PC3, and 5×10⁵ cells/insert for LNCaP) in MEM or RPMI containing 0.2% BSA (Sigma-Aldrich, St. Louis, MO). Chemoattractant solution, used as positive control, was made by diluting the epidermal growth factor (EGF) (10 ng/ml) into MEM (PC3 and DU145) or RPMI (LNCaP) supplemented with 0.2% BSA. Media containing 0.2% BSA served as a control. Control and EGF solutions (400 l) were added into the wells of a 24-well plate. Aliquots of 100 μl of cell suspensions, treated with or without the different compounds at several concentrations, were loaded into the transwell inserts and the plates were incubated at 37° C. for 5 hours (DU145 and PC3) or 24 hours (LNCaP). The cells inside the transwell inserts were removed by cotton swabs, and the cleaned inserts were fixed with 350 μl of 4% paraformaldehyde (pH 7.5) for 20 minutes at room temperature. Cells that had migrated to the outside of the transwell insert membrane were stained with 3 ng/ml of DAPI solution (Thermo Fisher Scientific, Eugene, OR) and images of five non-overlapping fields were captured using Axiovert 200M, Carl Zeiss (Thornwood, NY) microscope. The number of stained nuclei were determined with automatic counting using image analysis software (ZEN 2012; Carl Zeiss). Results were expressed as migration index defined as the average number of cells per field for test substance/the average number of cells per field for the medium control.

### Western Blot Analysis

Western blot analyses were performed as described previously (Caggia et al., “Novel Role of Gialpha2 in Cell Migration: Downstream of PI3-kinase-AKT and Rac1 in Prostate Cancer Cells,” J Cell Physiol, 234:802-815 (2018)). Briefly, cell lysates were mixed with Laemmeli's buffer (62.5 mM Tris, pH 6.8, 2% sodium dodecyl sulfate, 5% β-mercaptoethanol, and 10% glycerol). Samples (30-35 μg proteins) were separated on 10% SDS-PAGE gels and transferred to polyvinylidene difluoride (PVDF) membranes (Millipore Corp., Bedford, MA, USA). After blocking the membranes with 5% fat-free milk in TBST (50 mM Tris, pH 7.5, containing 0.15 M NaCl, and 0.05% Tween 20) for 1 hour at room temperature, the membranes were incubated with specific primary antibodies, at appropriate dilutions (1:5000 for Gαᵢ2; 1:3000 for α-tubulin) overnight at 4° C. After washing, the blots were incubated with appropriate secondary antibodies. The blots were then developed in Millipore Luminata Forte (EMD Millipore) for 5 min and visualized using BioRad ChemiDoc Imaging System, according to the manufacturer's instructions. The density of specific protein bands was determined using ImageJ image analyses software.

### Statistical Analysis

All experiments were repeated at least three times using different cell preparations, and two biological replicates were used for each condition. The results are presented as mean±SEM. One-way ANOVA was employed to assess the significance of differences among various treatment groups (p<0.05), using SigmaPlot software.

### Synthesis of Exemplifying Compounds

Representative procedure for the synthesis of ketimines

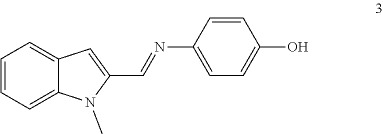 [SMILES: Cn1c(/C=N/c2ccc(O)cc2)cc2ccccc21]

Synthesis of (E)-4-(((1-methyl-1H-indol-2-yl)methylene)amino)phenol (3): A mixture of 1-methyl-1H-indole-2-carbaldehyde 1a (483 mg, 3.14 mmol), 4-aminophenol 2a (220 mg, 2.02 mmol) and glacial acetic acid (0.2 mL) in dry EtOH (10 mL) was heated in a pressure tube at 95° C. for 1.5 hours during which TLC revealed a quantitative consumption of 2a. Solvent was evaporated and the reddish paste was co-evaporated with CH₂Cl₂ (3×15 mL). The residue was suspended in Et₂O/CH₂Cl₂ 6:4 (20 mL) and kept in the refrigerator overnight. The suspension was filtered and the residue was copiously washed with ambient temperature Et₂O (ca. 60 mL). The brick-red precipitate was air-dried and then completely dried in vacuo. The filtrate was evaporated off and the reddish paste was suspended in Et₂O/CH₂Cl₂ 7:3 (20 mL) and kept in the refrigerator overnight. The suspension was filtered to give a second crop of the desired compound 3. Combined yield=270 mg (1^{st} crop)+30 mg (2^{nd} crop)=300 mg (59%). ¹H NMR (700 MHz, DMSO-d6) δ 9.52 (s, 1H), 8.71 (s, 1H), 7.64 (d, J=8.4 Hz, 1H), 7.52 (d, J=7.6 Hz, 1H), 7.28 (t, J=7.6 Hz, 1H), 7.24 (d, J=8.5 Hz, 2H), 7.09 (t, J=7.5 Hz, 1H), 6.82 (d, J=8.9 Hz, 2H), 4.17 (s, 3H). ¹³C NMR (176 MHz, DMSO-d6) δ 156.5, 149.6, 143.0, 139.9, 136.0, 126.9, 124.2, 122.6, 121.7, 120.3, 116.0, 110.5, 109.7, 31.8. HRMS (EI) m/z Calcd. for C₁₆H₁₅ON₂ [M+H]⁺: 251.1179, found 251.1177.

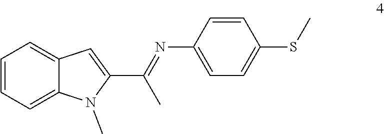 [SMILES: CSc1ccc(/N=C(\C)c2cc3ccccc3n2C)cc1]

(E)-1-(1-methyl-1H-indol-2-yl)-N-(4-(methylthio)phenyl)ethan-1-imine (4): The reaction of 1-(1-methyl-1H-indol-2-yl)ethan-1-one 1b (154 mg, 0.89 mmol), 4-(methylthio)aniline 2b (0.5 mL, 3.9 mmol) and glacial acetic acid (0.1 mL) (without EtOH) in a pressure tube under reflux (105-110° C.) within 24 hours, followed by purification on silica gel column chromatography eluting with EtOAc/Hexanes 1:8, gave 4 as light-yellow solid. Yield=146 mg (56%). HRMS (EI) m/z Calcd. for C₁₈H₁₈SN₂ [M+H]⁺: 295.1263, found 295.1261.

Representative Procedure for the Reduction of Ketimines with NaBH₄

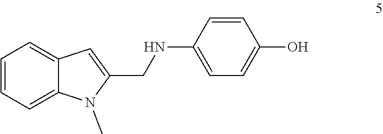 [SMILES: Cn1c(CNc2ccc(O)cc2)cc2ccccc21]

Synthesis of 4-(((1-methyl-1H-indol-2-yl)methyl)amino)phenol (5): To a solution of 3 (120 mg, 0.48 mmol) in MeOH (10 mL) was added NaBH₄ (75 mg, 1.92 mmol) upon which a rapid bubbling occurred. Stirring continued at room temperature for 45 min during which TLC revealed a quantitative conversion to a lower retention factor (rf) product. Water (30 mL) was added and the milky suspension was extracted with CH₂Cl₂ (4×20 mL). The combined organic layer was washed with brine (30 mL) and dried over Na₂SO₄. Solvent was evaporated off to give 5 as a brownish white solid. Yield=113 mg (93%). ¹H NMR (400 MHz, CDCl3) δ 7.57 (d, J=7.9 Hz, 1H), 7.31 (d, J=8.2 Hz, 1H), 7.24-7.19 (m, 1H), 7.12-7.07 (m, 1H), 6.71 (d, J=8.7 Hz, 2H), 6.62 (d, J=8.9 Hz, 2H), 6.45 (s, 1H), 4.36 (s, 2H), 3.74 (s, 3H). ¹³C NMR (176 MHz, CDCl3) δ 149.3, 149.3, 141.3, 137.8, 137.7, 127.4, 121.3, 120.3, 120.2, 119.3, 116.0, 115.0, 109.0, 100.4, 41.8, 29.6.

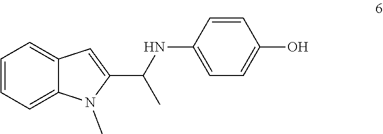 [SMILES: CC(Nc1ccc(O)cc1)c1cc2ccccc2n1C]

4-((1-(1-methyl-1H-indol-2-yl)ethyl)amino)phenol (6): The reduction of 14 (110 mg, 0.42 mmol) with NaBH₄ (120 mg, 3.2 mmol) in MeOH (15 mL), as described for compound 5, furnished the desired compound 6 as a reddish-white solid. Yield=110 mg (99%). ¹H NMR (400 MHz, CDCl3) δ 7.56 (dd, J=7.9, 1.0 Hz, 1H), 7.32-7.28 (m, 1H), 7.20 (t, J=7.6 Hz, 1H), 7.08 (t, J=7.9 Hz, 1H), 6.67 (d, J=8.5 Hz, 2H), 6.54 (d, J=8.6 Hz, 2H), 6.46 (t, J=0.8 Hz, 1H), 4.69 (q, J=6.5 Hz, 1H), 3.73 (s, 3H), 1.62 (d, J=6.5 Hz, 3H). ¹³C NMR (176 MHz, CDCl3) δ 148.0, 142.8, 141.2, 138.0, 127.4, 121.5, 120.6, 119.6, 116.4, 115.1, 109.1, 98.8, 47.1, 30.0, 20.5. MS [M+H]⁺ 267.1.

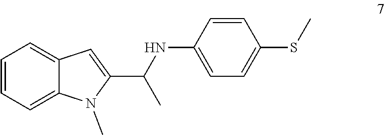 [SMILES: CSc1ccc(NC(C)c2cc3ccccc3n2C)cc1]

N-(1-(1-methyl-1H-indol-2-yl)ethyl)-4-(methylthio)aniline (7): The reduction of 4 (100 mg, 0.34 mmol) with NaBH₄ (120 mg, 3.2 mmol) in MeOH (10 mL) and CH₂Cl₂ (5 mL), as described for compound 5, after purification by prep-TLC eluting with Hexanes/EtOAc 7:1, furnished the desired compound 7 as a solid. Yield=55 mg (55%). MS [M+H]⁺ 297.2.

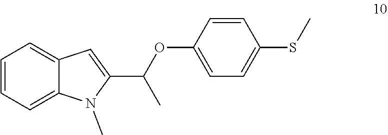 [SMILES: CSc1ccc(OC(C)c2cc3ccccc3n2C)cc1]

1-methyl-2-(1-(4-(methylthio)phenoxy)ethyl)-1H-indole (10): Compound 1b (556 mg, 3.2 mmol) was treated with NaBH₄ (488 mg, 12.84 mmol) in MeOH (30 mL) at room temperature for 45 min during which TLC revealed a quantitative formation of a lower retention factor (rf) product (rf=0.24, EtOAc/Hexanes 1:5). The reaction was worked up as described for the synthesis of 5 and the resulting crude 1-(1-methyl-1H-indol-2-yl)ethan-1-ol 8 was used without further purification.

A solution of compound 8 (440 mg, 2.51 mmol), triphenylphosphine (730 mg, 2.76 mmol), 4-(methylthio)phenol 9a (400 mg, 2.76 mmol), and diisopropyl azodicarboxylate (DIAD) (0.56 mL, 2.76 mmol) in tetrahydrofuran (15 mL) was stirred at room temperature for 24 hours. The reaction was partitioned between sat NaHCO₃ (40 mL) and EtOAc (40 mL). The two layers were separated, and the organic layer was washed with sat NaHCO₃ (30 mL), brine (30 mL) and dried over Na₂SO₄. Solvent was evaporated off and the crude was purified on silica gel chromatography, eluting with EtOAc/Hexanes 1:9 to give 10 as a white solid. Yield=315 mg (42%). ¹H NMR (700 MHz, CDCl₃) δ 7.61 (d, J=7.8 Hz, 1H), 7.33 (d, J=8.3 Hz, 1H), 7.23 (m, 3H), 7.12 (t, J=7.5 Hz, 1H), 6.94 (d, J=8.9 Hz, 2H), 6.56 (s, 1H), 5.61-5.58 (m, 1H), 3.79 (s, 3H), 2.45 (s, 3H), 1.82 (d, J=6.5 Hz, 3H). ¹³C NMR (176 MHz, CDCl₃) δ 155.9, 139.4, 138.1, 129.9, 129.6, 127.2, 121.9, 120.8, 119.6, 116.7, 109.1, 100.6, 69.8, 30.4, 20.1, 17.8. HRMS (EI) m/z Calcd. for C₁₈H₁₉OSN [M+H]⁺: 298.1260, found 298.1256.

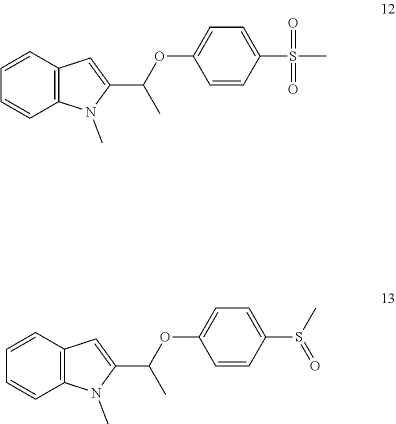 [SMILES: CC(Oc1ccc(S(C)(=O)=O)cc1)c1cc2ccccc2n1C ; CC(Oc1ccc(S(C)=O)cc1)c1cc2ccccc2n1C]

1-methyl-2-(1-(4-(methylsulfonyl)phenoxy)ethyl)-1H-indole (12) and 1-methyl-2-(1-(4-(methylsulfinyl)phenoxy)ethyl)-1H-indole (13): To a solution of 10 (70 mg, 0.24 mmol), diisopropylamine (6.6 L, 0.05 mmol), in CH₃CN (1 mL) was added dropwise to a solution of oxone (232 mg, 0.38 mmol) in water (2.0 mL). Stirring continued at room temperature and a precipitate developed. Ice cold water (15 mL) was added, and the suspension was filtered. The residue was washed with cold water (30 mL) and air dried. TLC of the dried solid revealed a quantitative formation of 10 and formation of two new products (rfs 0.77 and 0.45, CH₂Cl₂/MeOH 14:1). The crude was purified with prep-TLC, eluting with CH₂Cl₂/MeOH 20:1 to furnish 12 (rf=0.77 compound) and 13 (rf=0.45 compound) as white solids. Yield: compound 12=14 mg (18%); compound 13=16 mg (22%).

Characterization of 12: ¹H NMR (700 MHz, CDCl₃) δ 7.85 (d, J=6.7 Hz, 2H), 7.62 (d, J=7.9 Hz, 1H), 7.34 (d, J=8.3 Hz, 1H), 7.28-7.24 (m, 3H), 7.12 (d, J=7.9 Hz, 2H), 5.73 (q, J=6.5 Hz, 1H), 3.79 (s, 3H), 3.02 (s, 3H), 1.88 (d, J=6.9 Hz, 3H). ¹³C NMR (176 MHz, CDCl₃) δ 161.7, 138.2, 138.2, 132.7, 129.6, 127.1, 122.2, 120.9, 119.9, 116.0, 109.2, 101.1, 70.3, 44.8, 30.5, 20.4. Calcd. for C₁₈H₁₉O₃SN [M+H]⁺: 330.1158, found 330.1144.

Characterization of 13: ¹H NMR (700 MHz, CDCl₃) δ 7.61 (d, J=8.0 Hz, 1H), 7.57 (dd, J=8.9, 3.1 Hz, 2H), 7.33 (d, J=8.3 Hz, 1H), 7.25 (t, J=7.6 Hz, 2H), 7.13 (d, J=8.8 Hz, 2H), 6.58 (s, 1H), 5.69 (q, J=6.5 Hz, 1H), 3.80 (s, 3H), 2.71 (s, 3H), 1.86 (dd, J=6.6, 3.3 Hz, 3H). 13C NMR (176 MHz, CDCl3) δ 160.0, 159.9, 138.1, 129.7, 127.1, 125.6, 125.6, 122.1, 120.8, 119.8, 116.6, 109.1, 100.9, 100.9, 70.0, 43.9, 30.4, 20.3. Calcd. for C₁₈H₁₉O₂SN [M+H]⁺: 314.1209, found 314.1198.

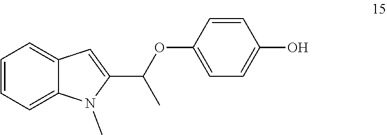 [SMILES: CC(Oc1ccc(O)cc1)c1cc2ccccc2n1C]

4-(1-(1-methyl-1H-indol-2-yl)ethoxy)phenol (15): Compound 8 (330 mg, 1.9 mmol), triphenylphosphine (608 mg, 2.30 mmol), 4-((tert-butyldimethylsilyl)oxy)phenol 9b (516 mg, 2.30 mmol), and diisopropyl azodicarboxylate (DIAD) (0.46 mL, 2.30 mmol) in tetrahydrofuran (10 mL) was stirred at room temperature for 24 hours. The reaction was worked up as described for the synthesis of 10 to furnish the crude compound 11, which was subsequently reacted with CsF (364 mg, 2.4 mmol) in MeOH (20 mL) at room temperature for 2 hours. The reaction was partitioned between water (40 mL) and CH₂Cl₂ (50 mL). The two layers were separated, the aqueous layer was washed with CH₂Cl₂ (40 mL), the combined organic layer was washed with half-saturated brine (50 mL) and dried over Na₂SO₄. Solvent was evaporated off and the crude was purified on prep-TLC, eluting with EtOAc/Hexanes 1:4 to give 15 as a white solid. ¹H NMR (700 MHz, CDCl₃) δ 7.63-7.57 (d, J=7.9 Hz, 1H), 7.35-7.31 (d, J=8.3 Hz, 1H), 7.26-7.21 (d, J=8.2 Hz, 1H), 7.15-7.10 (t, J=7.5 Hz, 1H), 6.88-6.82 (s, 2H), 6.75-6.68 (s, 2H), 6.52 (s, 1H), 5.52-5.47 (q, J=6.6 Hz, 1H), 4.6 (bs, 1H), 3.79 (s, 3H), 1.81-1.78 (d, J=6.6 Hz, 3H). ¹³C NMR (176 MHz, CDCl₃) δ 151.5, 150.0, 139.8, 138.1, 127.2, 121.8, 120.7, 119.5, 117.7, 116.1, 109.1, 100.5, 70.2, 30.4, 20.2. MS [M+H]⁺268.4.

### Example 2—Optimization of the First Generation Gαᵢ2 Inhibitors

Inventors have disclosed previously that compound 14 attenuated the EGF-induced migration of prostate cancer cell lines PC3 and DU-145 (Caggia et al., “Small Molecule Inhibitors Targeting Galphai2 Protein Attenuate Migration of Cancer Cells,” Cancers (Basel), 12(6):1631 (2020)). However, 14 is a phenolic imine, which is sometimes more stable than other imines (Al Zoubi et al., “Acyclic and Cyclic Imines and Their Metal Complexes: Recent Progress in Biomaterials and Corrosion Applications,” RSC Advances, 8:23294-23318 (2018); Kumar et al., “A Comprehensive Review on Pharmacological Activity of Schiff Base Containing Derivatives,” Organic & Medicinal Chem IJ, 1(3):555564 (2017)), but could still be beleaguered by metabolic liability in in vivo settings. Hence, inventors first set out to identify analogs that may be devoid of metabolically liable moieties but with improved or comparable potency as 14. Molecular docking analysis, using Autodock Vina (Trott and Olson, “AutoDock Vina: Improving the Speed and Accuracy of Docking with a New Scoring Function, Efficient Optimization, and Multithreading. J Comput Chem, 31:455-461 (2010)) on the structure Gαᵢ-GDP (PDB: 20M2), revealed that 14 adopted docked pose that suggests its imine moiety could be replaced with a more stable amino or ether group while modifications that introduced steric hindrance at the phenolic moiety may not be so well tolerated due to the need to accommodate Mg²⁺ ion at the binding site. Based on this observation, compounds 3-7, 10, 12-13 and 15, analogs of 14 modified within the aforementioned moieties and c-methyl group were designed. These compounds were facilely synthesized as shown in Scheme 1. Briefly, reaction of 1a-b with 2a-b and glacial acetic acid in EtOH at 95° C. furnished imines 3-4 and the previously disclosed compound 14. The reduction of 3-4 and 14 with NaBH₄ in MeOH resulted in secondary amines 5-7 as racemic mixtures. Similarly, NaBH₄ reduction of 1b furnished compound 8, which was subjected to Mitsunobu reaction (Manhas et al, “Steroids. Part X. A Convenient Synthesis of Alkyl Aryl Ethers,” Journal of the Chemical Society, Perkin Transactions, 1:461-463 (1975)) with 9a-b to furnish ether compounds 10-11. Oxidation of the thiomethyl group of 10 by treatment with oxone (Kupwade et al., “Catalyst-free Oxidation of Sulfides to Sulfoxides and Diethylamine Catalyzed Oxidation of Sulfides to Sulfones using Oxone as an Oxidant,” Research on Chemical Intermediates, 43:6875-6888 (2017)) afforded methylsulfone 12 and methylsulfoxide 13 as racemic and diasteromeric mixtures respectively. The TBDMS group of 11 was removed using CsF to afford compound 15 as a racemic mixture.

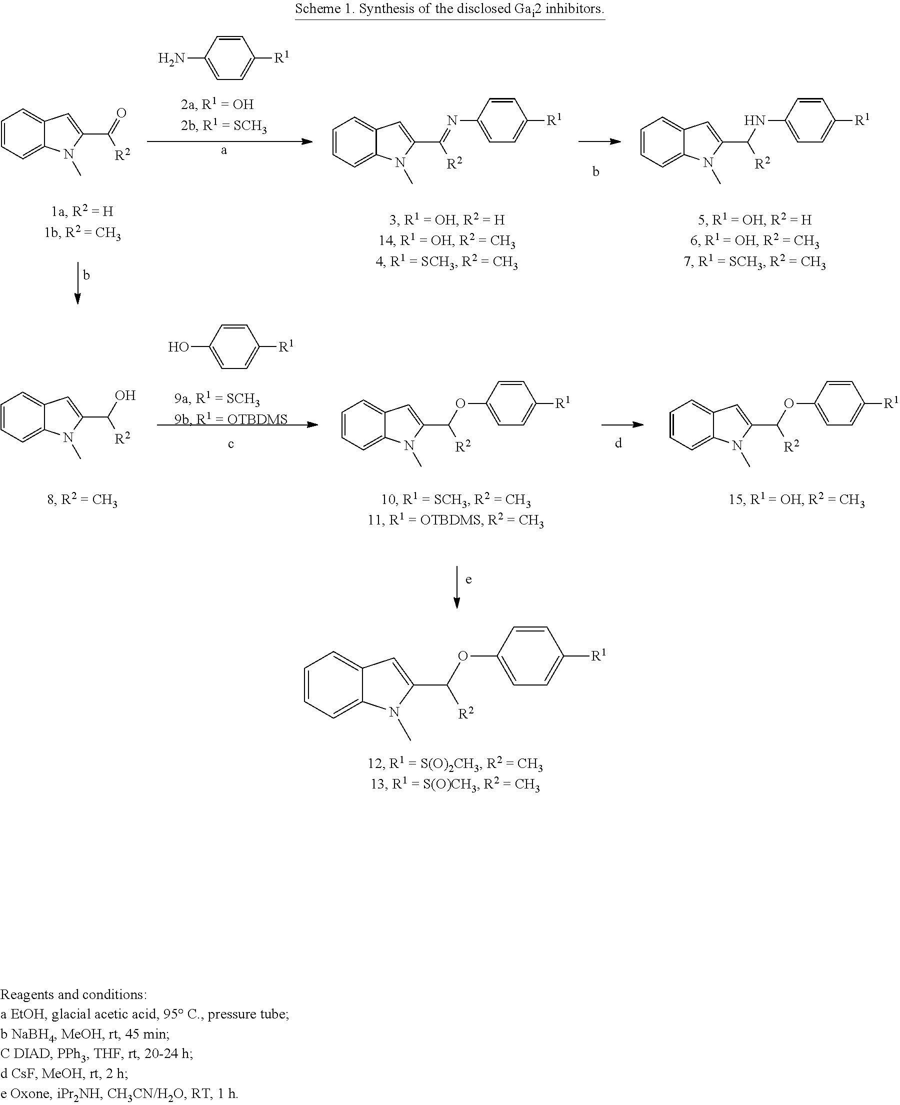

Scheme 2 provides a synthetic scheme for proposed compounds 21-S and 21-R.

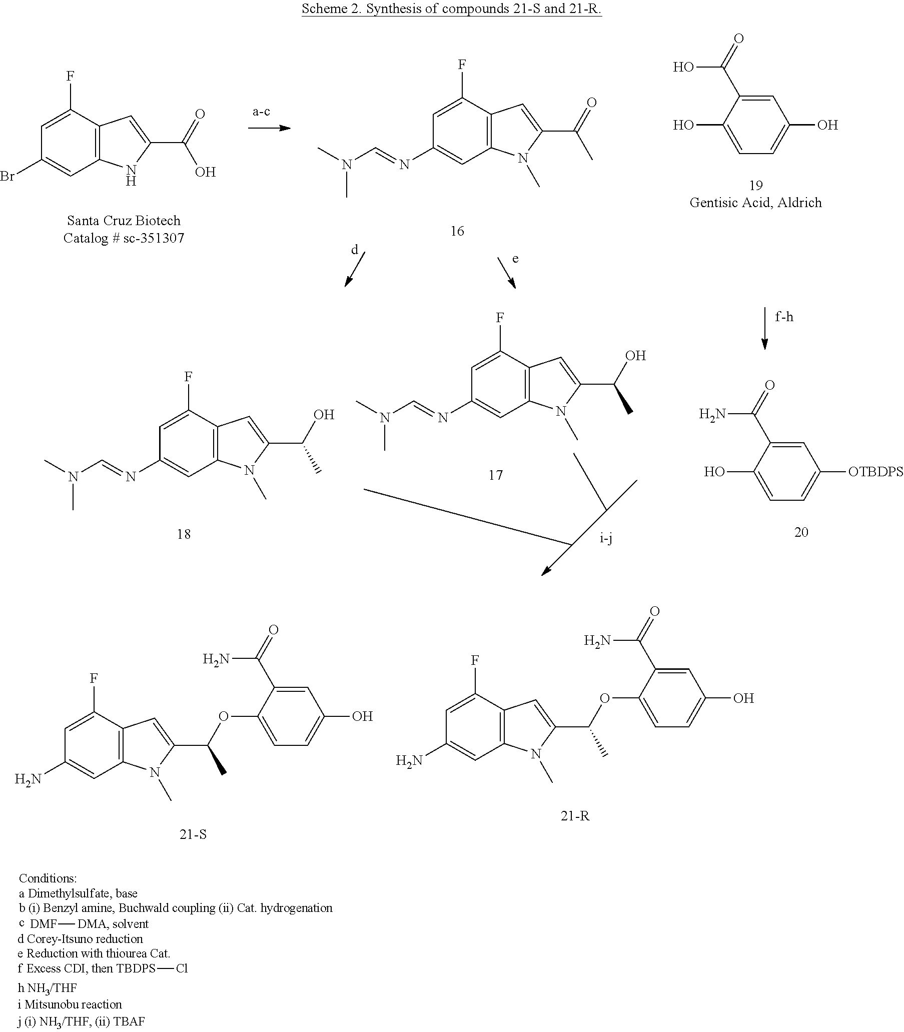

### Example 3—Effect of New Compounds on the Migration of Prostate Cancer Cell Lines

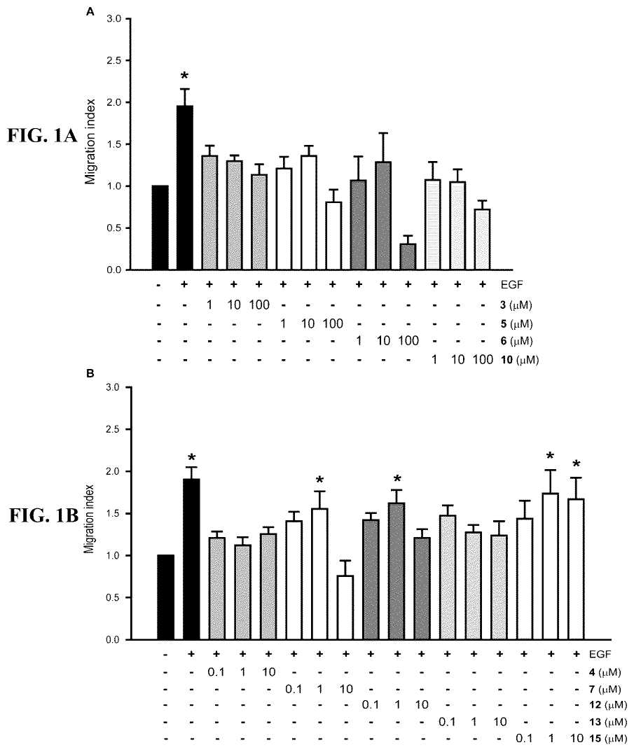

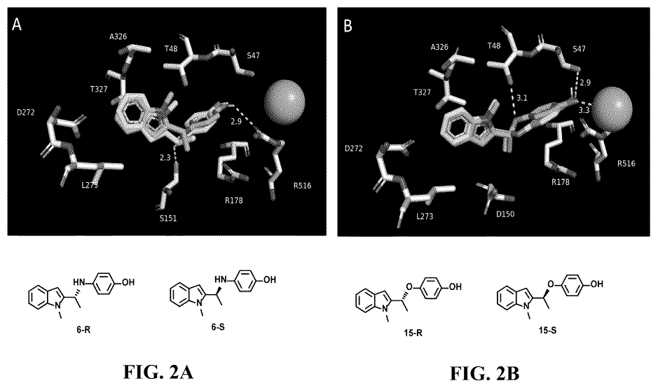

The effects of the newly synthesized compounds on the migratory behavior of PC3 cells were investigated in transwell migration assays as inventors have done before (Caggia et al., “Small Molecule Inhibitors Targeting Galphai2 Protein Attenuate Migration of Cancer Cells,” Cancers (Basel), 12(6):1631 (2020)). Compounds were tested at a concentration range of 0.1 to 100 μM. The new imine compounds 3 and 4 caused a concentration dependent reduction of PC3 cell migration, but less efficiently relative to 14. Interestingly, the secondary amines 5-7 more efficiently attenuated PC3 migration, with 5 having comparable potency and compounds 6 and 7 more effective relative to 14. The ether compounds 10, 12, 13 and 15 displayed varying effect on PC3 migration. Compound 10, an analog in which the amino group for 7 has been switched to an ether group is less potent compared to 7. This suggests that the accommodation within the active site of Gαi-GDP of the thiomethyl moiety, common to both compounds, is highly dependent on the type of moiety at the para-position connecting to the methylindole group. The methylsulfone 12 and methylsulfoxide 13 are less effective, eliciting reduced anti-migration activities relative to the other compounds (FIG. 1). Surprisingly, compound 15, an ether analog of the potent amine 6, did not inhibit the migration of PC3 cells at the maximum concentration tested (FIG. 1). To potentially obtain insight into the disparity in their Gαi inhibition activities, molecular docking was performed on the enantiomers of 6 (6-R and 6-S) and 15 (15-R and 15-S). It was observed that the enantiomeric pair 6-R and 6-S bind to Gαi with slightly better binding energies than 14, while adopting similar orientation (FIG. 2). Additionally, 6-S seems to have slightly improved binding affinity relative to 6-R. The enantiomeric pair 15-R and 15-S are also well accommodated within the Gαi active site with the same binding affinity. However, their binding affinity is slightly weaker than that of 6-R and 6-S(FIG. 2). This slightly weaker binding affinity may partially explain the disproportionately weaker potency of 15 relative to 6. Other molecular factors, such as those which led to the attenuated potency observed with amino- to ether-group switch in the 7/10 pair, may contribute to the reduced potency of 15. Finally, most of these compounds have no effects on the morphology of PC3 cells or viability of several cancer cells at the concentrations as high as 100 μM.

### Example 4—Evaluation of In Vitro Stability of Representative Compound

On the strength of their potency, the stability of 6, 7 and 10 was determined in human and mouse plasma and liver microsomes. It was observed that these compounds are stable in the plasma of either specie, with half-life ranging from 27 min to >120 min. In the liver microsomes, the stability of these compounds is highly specie dependent with 6 robustly stable in both species while 7 and 10 are only stable in the human microsome (Table 1).

TABLE 1
Stability of compounds 6, 7 and 10 in human
and mouse plasma and liver microsomes

| | Plasma (t_{1/2} = Min) | | Microsome (t_{1/2} = Min) | |
|---|---|---|---|---|
| Compound | Human | Mouse | Human | Mouse |
| 6 | >120 | 27 | 118 | 47 |
| 7 | >120 | >120 | 45 | <10 |
| 10 | >120 | >120 | 35 | <10 |

### Example 5—HDACi Induce Cell Migration in LNCaP Prostate Cancer Cell Lines

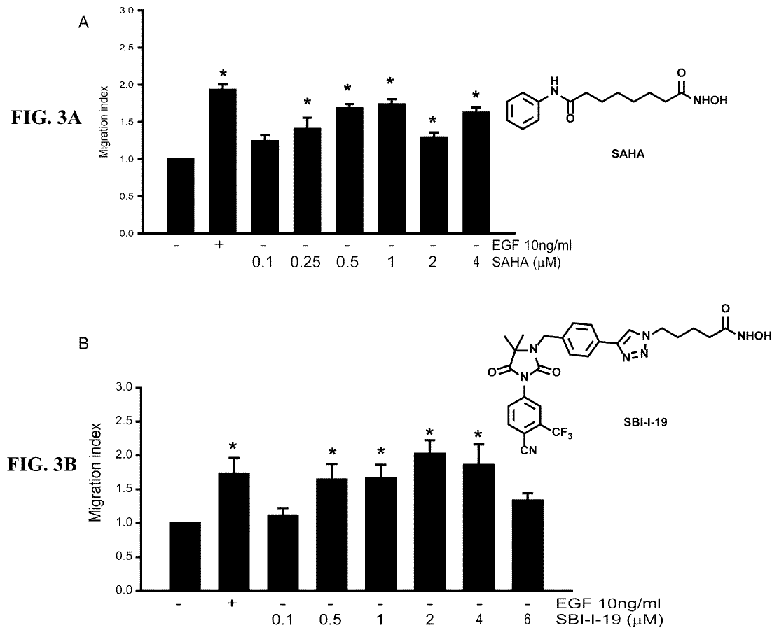

To determine the effects of HDACi on prostate cancer cell migration, migration assays were performed in androgen receptor (AR)-positive LNCaP cells using several concentrations of SAHA (0.1, 0.25, 0.5, 1, 2, 4 μM), a commercially available HDACi. As shown in FIG. 3A, treatments with SAHA induced a significant dose-dependent increase in cell migration. Maximum induction of cell migration was observed at 0.5 and 1 μM doses of SAHA. Inventors have previously developed a very potent HDACi inhibitor (SBI-I-19), coupled with an anti-androgen, enzalutamide, which selectively targets androgen sensitive prostate cancer cells for its cytotoxic effects (Gryder et al., “Selectively Targeting Prostate Cancer with Antiandrogen Equipped Histone Deacetylase Inhibitors,” ACS Chem Biol, 8:2550-2560 (2013)). The possible effects of different concentrations of SBI-I-19 on cell migration in LNCaP cells were determined. As shown in FIG. 3B, SBI-I-19 also induced significant dose-dependent increase in cell migration, when compared to the untreated cells, with maximum effects observed at 1 and 2 μM.

### Example 6—Anti-Androgens Induce Cell Migration in AR− Positive LNCaP Prostate Cancer Cells

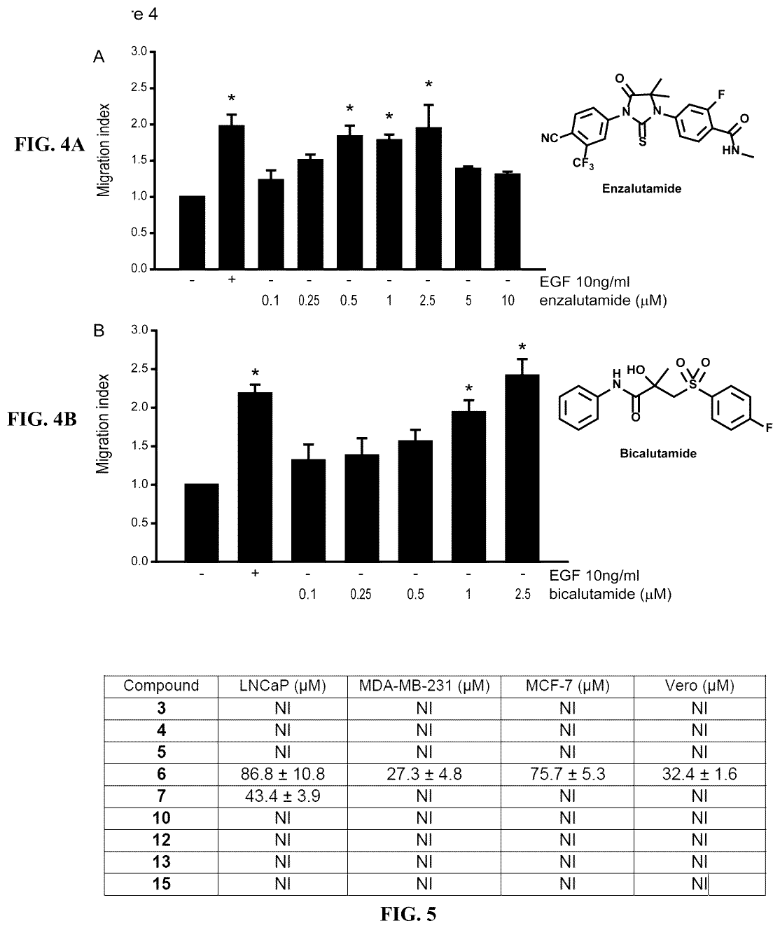

Since SBI-I-19 was equipped with an anti-androgen moiety inspired by enzalutamide, the observed effects on cell migration could be due to its anti-androgen moiety. To investigate this possibility, LNCaP cells were treated with different concentrations of two FDA approved anti-androgen drugs (enzalutamide and bicalutamide). Surprisingly, as shown in FIGS. 4A and 4B, both anti-androgens induced a dose-dependent increase in cell migration in LNCaP cells. The maximum effects of both compounds were observed at a dose of 2.5 μM.

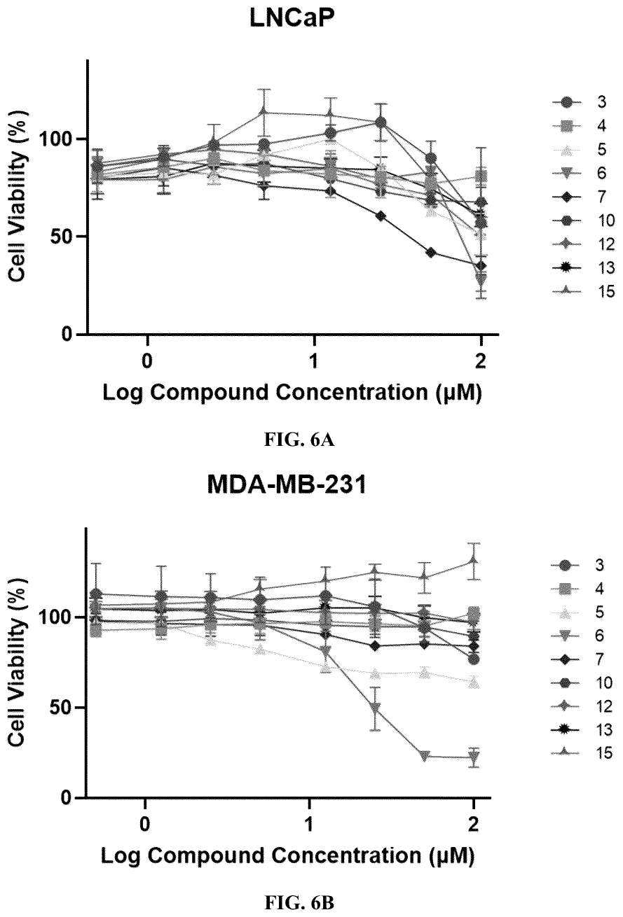

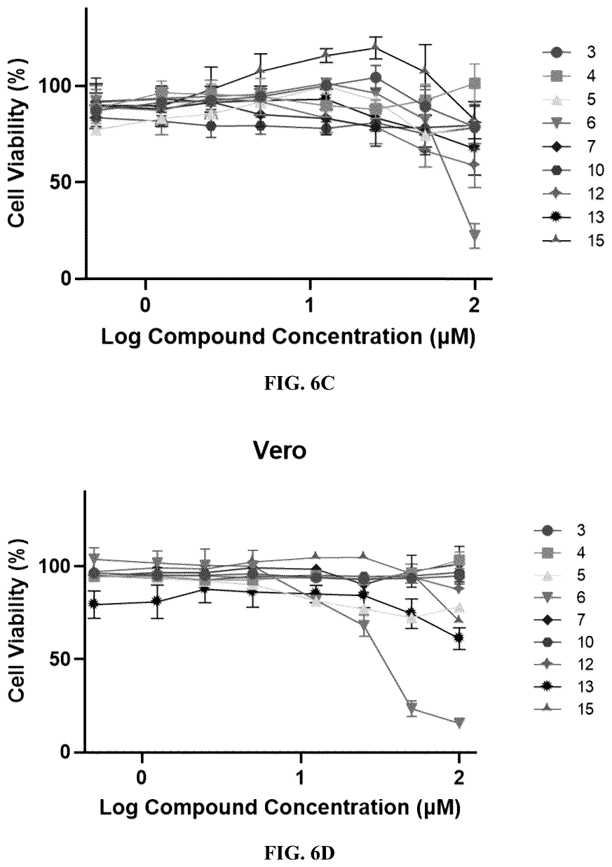

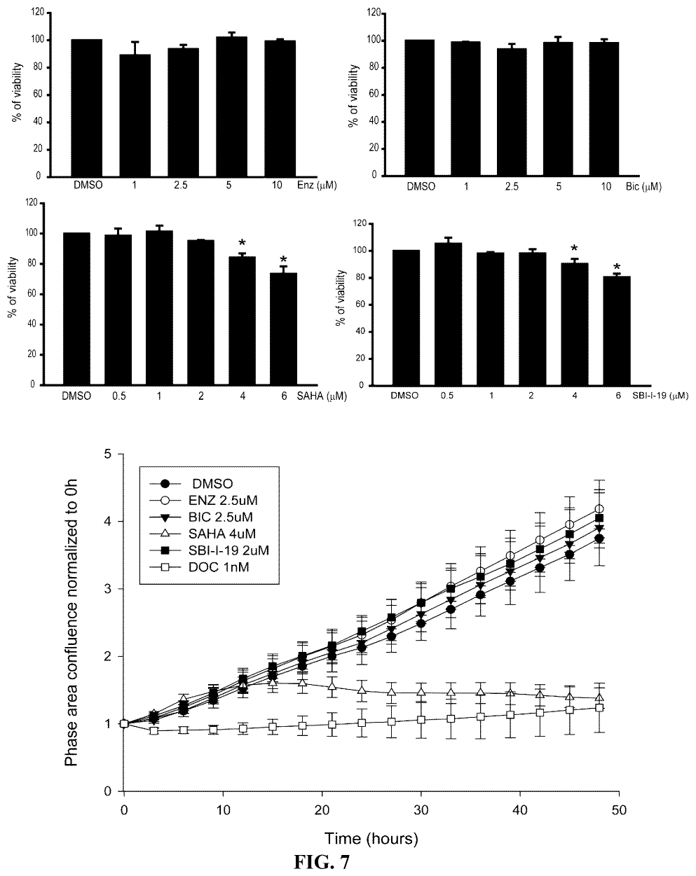

To determine whether the chemotherapeutic drugs affected cell viability and proliferation, LNCaP cells were treated with different concentration of the drugs, and performed MTS assays and proliferation assays, using Incucyte SX5 system (FIGS. 5, 6A-6D, 7). The drugs did not significantly affect the proliferation of LNCaP cells.

### Example 7—Effects of Chemotherapy on Cancer Cell Migration in AR− Negative DU145 and PC3 Cells

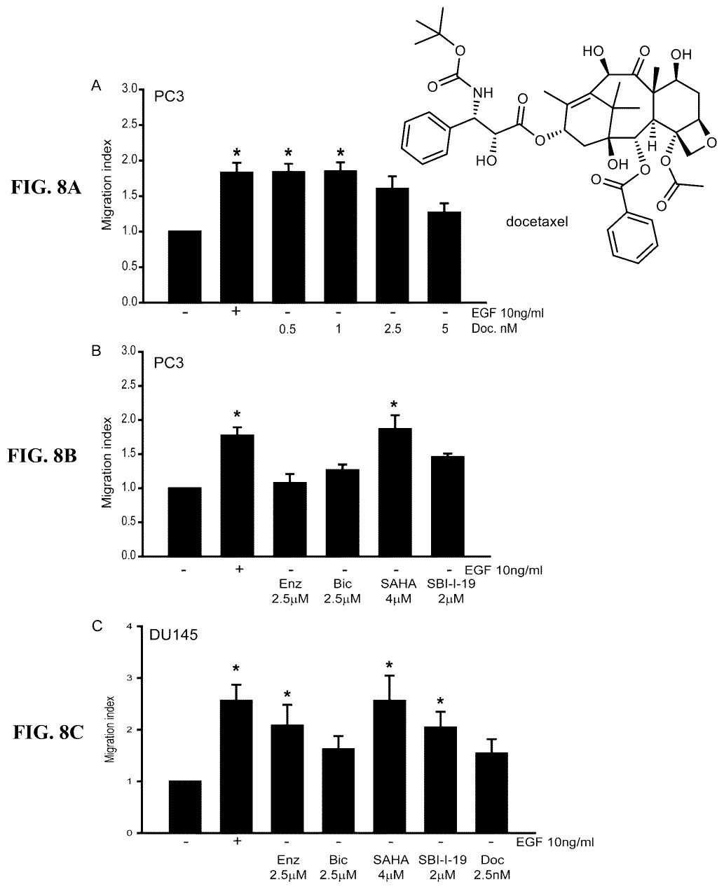

To determine whether the induction of cell migration by AR-antagonists and HDACi was limited to only AR-positive cells, the effects of anti-androgens and HDACi and docetaxel in AR-negative DU145 and PC3 cells were determined. Both antiandrogens did not have any effect on cell migration in PC3 cells (FIG. 8B). On the and other hand, treatments with HDACi (SAHA) or docetaxel, a chemotherapeutic drug commonly utilized to treat advanced castration-resistant metastatic prostate cancers, induced increase in cell migration in these cells (FIG. 8A-B). In DU145 cells, treatments with enzalutamide, and HDACi (SAHA and SBI-I-19) increased cell migration as well (FIG. 8C).

### Example 8—Anti-Androgens Upregulate the Expression of Gαᵢ2 Protein in LNCaP Cells

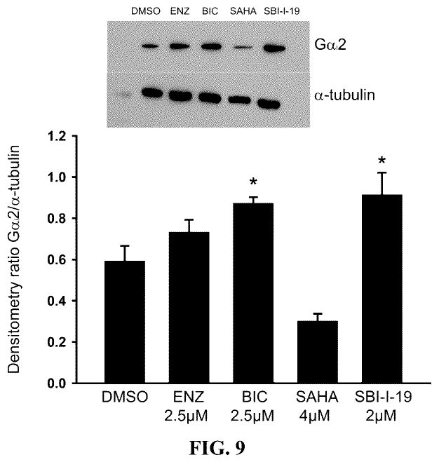

Previously inventors have shown the essential role of Gαᵢ2 protein in prostate cancer cell migration (Zhong et al., “The Essential Role of Gialpha2 in Prostate Cancer Cell Migration,” Mol Cancer Res, 10:1380-1388 (2012); Caggia et al., “Novel Role of Gialpha2 in Cell Migration: Downstream of PI3-kinase-AKT and Rac1 in Prostate Cancer Cells,” J Cell Physiol, 234:802-815 (2018)). To determine whether the HDACi and anti-androgens regulate the expression of Gαi2 protein, LNCaP cells were treated with anti-androgens and HDACi for 24 hours, using the most effective concentrations for each drug, and the levels of Gαᵢ2 protein were determined by Western blotting. As shown in FIG. 9, treatments with enzalutamide (2.5 μM), bicalutamide (2.5 μM) and SBI-I-19 (2 μM) resulted in increased levels of Gαᵢ2 protein in LNCaP cells. On the other hand, HDACi SAHA induced a reduction in the levels of Gαi2 protein. In PC3 cells, treatment with HDACi, anti-androgens or docetaxel (1 nM), had no effect on the levels of Gαᵢ2 protein (data not shown).

### Example 9—Gαᵢ2 Inhibitors Block the Effects of the Chemotherapeutic Drugs on Prostate Cancer Cell Migration

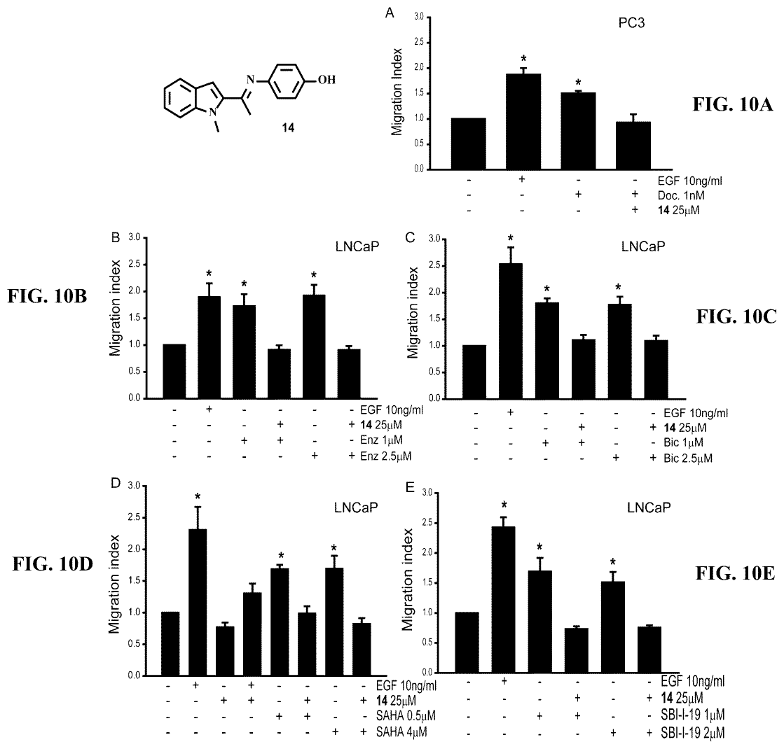

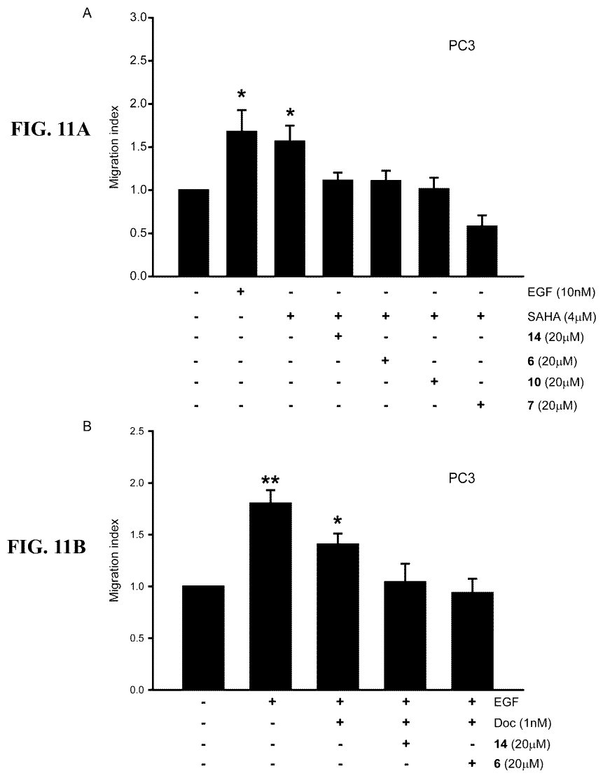

Inventors have recently developed small molecule inhibitors of Gαᵢ2 protein activation, which block cell migration and invasion in several cancer cell types (Caggia et al., “Small Molecule Inhibitors Targeting Galphai2 Protein Attenuate Migration of Cancer Cells,” Cancers (Basel), 12(6):1631 (2020)). In this study one of the most effective Gαᵢ2 inhibitors (compound 14; at 25 kM) was used in combination with the above chemotherapeutic drugs. As shown in FIGS. 10A-10E, simultaneous treatment with Gαᵢ2 inhibitor blocked the stimulatory effects of docetaxel in PC3 cells (FIG. 10A), and HDACi and anti-androgens effects on cell migration in LNCaP cells (FIGS. 10B-10E). Additionally, simultaneous treatments with the second generation Gαᵢ2 inhibitors (6, 7 and 10) blocked the stimulatory effects of docetaxel and SAHA in PC3 cells (FIGS. 11A-11B).

### Example 10—Discussion of Examples 2-9

The novel findings in this study show that anti-androgens and chemotherapeutic agents (docetaxel and HDACi) induce migratory behavior in prostate cancer cells and these effects on cell migration can be blocked by simultaneous treatment with a small molecule inhibitor of Gαᵢ2 protein.

Early-stage prostate cancers are localized in the prostate gland and are treatable by surgery and radiation therapy; the prognosis in these patients is very good. However, the prostate cancers in later stages of the disease metastasize to other tissues and bone, reducing drastically the overall survival of the patients (DeSantis et al., “Cancer Treatment and Survivorship Statistics,” CA: a cancer journal for clinicians, 64:252-271 (2014)). The current treatment option available to treat hormone-sensitive prostate cancers is androgen deprivation therapy (ADT), which is based on either inhibition of androgen biosynthesis or treatment with AR antagonists. In this study, it was observed that two of the most commonly used AR− antagonists, enzalutamide and bicalutamide, caused significant induction of cell migration in AR-positive prostate cancer cells, indicating that treatment with these drugs may lead to unintended consequences resulting in the development or aggravation of metastatic disease.

Interestingly, enzalutamide induced cancer cell migration in DU145 cells, which are AR-null prostate cancer cells. One possible explanation could be due to the fact that AR-negative DU145 prostate cancer cells have been shown to respond to enzalutamide treatments through the glucocorticoid receptor (GR), encoded by NR3C1 gene (Smith et al., “Enzalutamide Response in a Panel of Prostate Cancer Cell Lines Reveals a Role for Glucocorticoid Receptor in Enzalutamide Resistant Disease,” Scientific reports, 10:21750 (2020)).

Although chemotherapy is a standard treatment for several cancer types, tumors often recur at primary or distant sites following treatments (Miller et al., “Phase II Evaluation of Pemetrexed in the Treatment of Recurrent or Persistent Platinum-resistant Ovarian or Primary Reritoneal Carcinoma: A Study of the Gynecologic Oncology Group,” Journal of clinical oncology: official journal of the American Society of Clinical Oncology, 27:2686-2691 (2009); Holen and Saltz, “New Therapies, New Directions: Advances in the Systemic Treatment of Metastatic Colorectal Cancer,” The Lancet. Oncology, 2:290-297 (2001)). Several recent studies have shown that chemotherapeutic agents can promote cancer cell migration and participate in the development of metastatic disease (Dreaden et al., “Antiandrogen Gold Nanoparticles Dual-target and Overcome Treatment Resistance in Hormone-insensitive Prostate Cancer Cells,” Bioconjug Chem, 23:1507-1512 (2012); Ji et al., “HDAC Inhibitors Induce Epithelial-mesenchymal Transition in Colon Carcinoma Cells,” Oncol Rep, 33:2299-2308 (2015); Diaz-Nunez et al., “Histone Deacetylase Inhibitors Induce Invasion of Human Melanoma Cells In Vitro via Differential Regulation of N-cadherin Expression and RhoA Activity,” BMC Cancer, 16:667 (2016)). Due to the broad use of chemotherapeutic agents in cancer treatments, it is critical to investigate their metastasis-promoting activity (Liu et al., “Cisplatin-stimulated Macrophages Promote Ovarian Cancer Migration via the CCL20-CCR6 Axis,” Cancer letters, 472:59-69 (2020)). In the present study, treatment with HDACi and docetaxel resulted in increased migratory behavior in both AR-positive and AR-negative prostate cancer cells indicating that these effects on cell migration in prostate cancer cells are not limited to only anti-androgens. The mechanisms involved in these effects of various chemotherapies are not well understood. One of the possible events triggering chemotherapy-induced metastasis can be achieved by modulating non-cancer host cells and creating a favorable microenvironment for cancer cell dissemination (Gilbert and Hemann, “Chemotherapeutic Resistance: Surviving Stressful Situations,” Cancer Res, 71:5062-5066 (2011); Shiao et al., “Immune Microenvironments in Solid Tumors: New Targets for Therapy,” Genes & development, 25:2559-2572 (2011); Kitamura et al, “Immune Cell Promotion of Metastasis,” Nature Reviews Immunology, 15:73-86 (2015); Middleton et al., “Chemotherapy-Exacerbated Breast Cancer Metastasis: A Paradox Explainable by Dysregulated Adaptive-Response,” International Journal of Molecular Sciences, 19(11):3333 (2018)). Additionally, the cell cytotoxicity of the chemotherapeutic agents and/or hypoxia could potentially activate “escape” mechanisms, leading to cancer cell dissemination and metastasis (Zhang et al., “Perspectives on Hypoxia Signaling in Tumor Stroma,” Cancers (Basel), 13(12):3070 (2021); Filippi et al., “Short-term Hypoxia Enhances the Migratory Capability of Dendritic Cell through HIF-lalpha and PI3K/Akt Pathway,” J Cell Physiol, 229:2067-2076 (2014); Muz et al., “The Role of Hypoxia in Cancer Progression, Angiogenesis, Metastasis, and Resistance to Therapy,” Hypoxia, 3:83-92 (2015)). Whether these escape mechanisms involved induction of epithelial to mesenchymal transformation (EMT) and secretion of MMPs and other enzymes which lead to increased migratory and invasive behavior in these cells needs to be determined in future studies. However, these in vitro effects of anti-androgens and other agents (docetaxel, HDACi) were observed at very low doses, which did not exert any cytotoxic effects under the same experimental conditions (FIGS. 5, 6A-6D, 7). Therefore, the clinical relevance of these findings remains to be determined as the treatments with these agents in cancer patients are normally carried out at much higher cytotoxic doses.

In earlier studies, the inventors have shown that Gαᵢ2 protein plays an essential role in cell migration and invasion in prostate and other cancer cells and these effects of Gαᵢ2 protein are exerted at two distinct levels. One of these effects is dependent on the activation of GPCR signaling by specific ligands (such as SDF1α, PGE2) while the second effect is independent of GPCR signaling and is exerted at the level of lamellipodia formation in response to activation of multiple upstream pathways (Caggia et al., “Novel Role of Gialpha2 in Cell Migration: Downstream of PI3-kinase-AKT and RacI in Prostate Cancer Cells,” J Cell Physiol, 234:802-815 (2018)). In addition, inventors have also developed small molecule inhibitors of Gαᵢ2 which blocked migratory behavior of prostate and other cancer cells induced by various ligands (Caggia et al., “Novel Role of Gialpha2 in Cell Migration: Downstream of PI3-kinase-AKT and RacI in Prostate Cancer Cells,” J Cell Physiol, 234:802-815 (2018); Caggia et al., “Small Molecule Inhibitors Targeting Galphai2 Protein Attenuate Migration of Cancer Cells,” Cancers (Basel), 12(6):1631 (2020)). In the present study, it was observed that treatment with anti-androgens (enzalutamide and bicalutamide) induced an increase in intracellular levels of Gαᵢ2 protein in AR-positive LNCaP cells, but not in AR-negative PC3 cells, indicating that effects of anti-androgens on Gαᵢ2 levels may be mediated by AR. Increased Gαᵢ2 levels may, in turn, mediate increased cell migration, which also suggests a possible role of AR in regulation of cell migration. Additional studies are needed to explore this possibility. On the other hand, HDACi and Docetaxel had no effect on the expression levels of Gαᵢ2 protein indicating that the effects of these agents are distinct from anti-androgens. However, the essential role of Gαᵢ2 protein in cell migration was confirmed since inhibition of its activity by small molecule inhibitors resulted in attenuation of cell migration induced by all chemotherapeutic agents.

Currently, very few metastasis-specific therapeutic targets have been developed and effective prevention and suppression of metastatic disease is still an elusive goal (Fontebasso and Dubinett, “Drug Development for Metastasis Prevention,” Critical Reviews in Oncogenesis, 20:449-473 (2015)). Due to the essential role of Gαᵢ2 protein in cell migration and the ability of its inhibitors to block the effects of many different ligands on cell motility, Gαᵢ2 inhibitors could be promising anti-metastatic drug candidates. These findings also suggest that a combination therapy with Gαᵢ2 inhibitors along with anti-androgens and/or other chemotherapeutic agents may blunt the possible negative effects of these treatments in development of metastatic disease.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method comprising:
administering, to a subject with cancer, a chemotherapeutic drug under conditions effective to treat the subject for cancer; and
administering, to the subject, a G(alpha)i2 (Gαᵢ2) inhibitor.

2. The method of claim 1 wherein the Gαᵢ2 inhibitor is a compound of Formula I having the following structure: 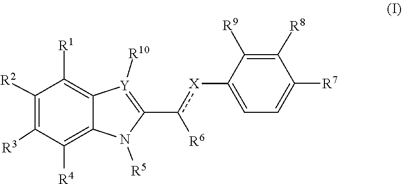
wherein [symbol]
is a single or double bond,
R¹, R², and R³ are each independently H, —OH, —NH₂, or halogen;
R⁴ is H, alkyl, halo-alkyl, aryl, pyridyl, wherein the aryl and pyridyl groups are optionally substituted at the ortho, meta, and para positions;
R⁵ is H, methyl, or ethyl;
R⁶ is H, methyl, or ethyl;
R⁷ is —OH, -OMe, -SMe, -SOMe, or —SO₂Me;
R⁸ and R⁹ are each independently H or —C(O)NH₂;
R¹⁰ is H, —OH, or halogen;
X is N or O; and
Y is C or N.

3. The method of claim 1, wherein the Gαᵢ2 inhibitor is selected from the group consisting of:

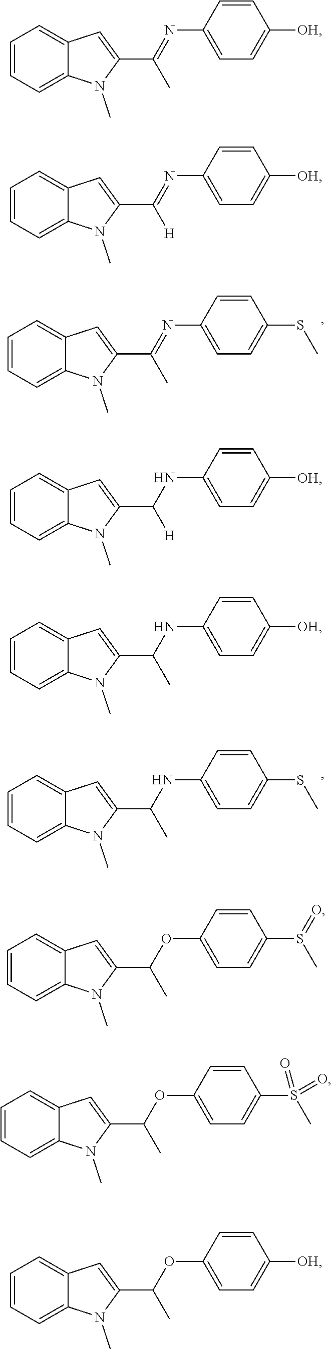

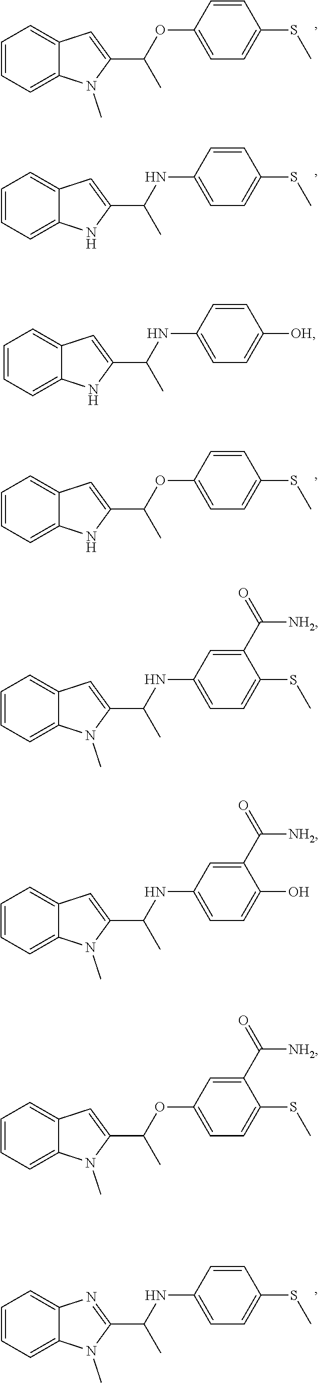

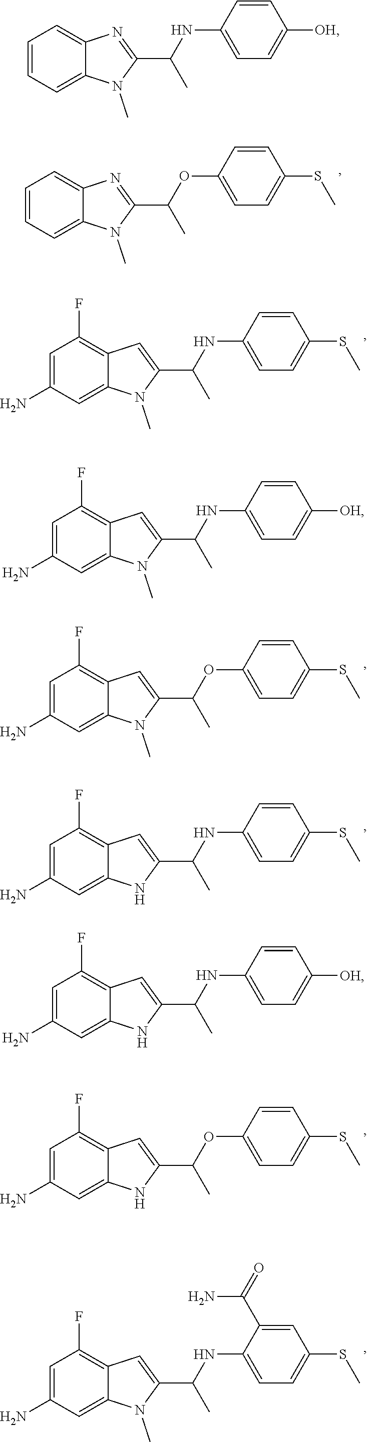

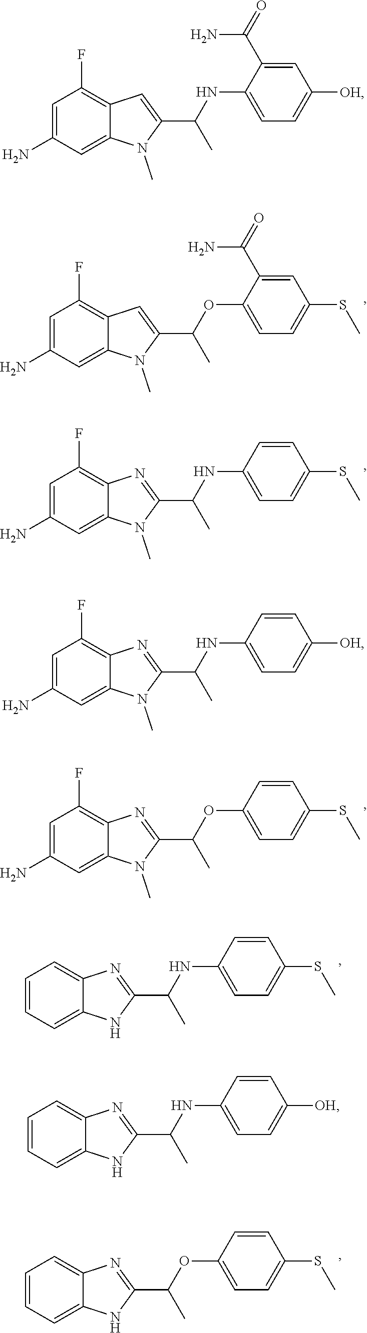

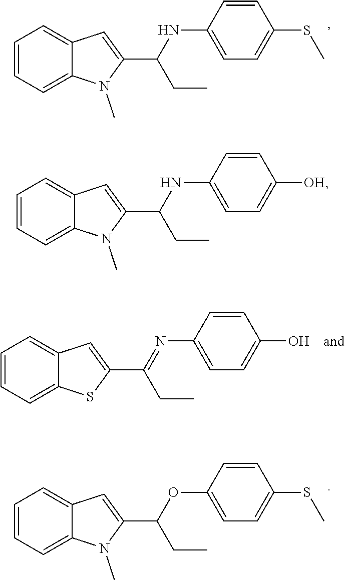 [SMILES: CCC(Nc1ccc(SC)cc1)c1cc2ccccc2n1C ; CCC(Nc1ccc(O)cc1)c1cc2ccccc2n1C ; CC/C(=N\c1ccc(O)cc1)c1cc2ccccc2s1 ; "and" ; CCC(Oc1ccc(SC)cc1)c1cc2ccccc2n1C]

4. The method of claim 2, wherein the Gαᵢ2 inhibitor is

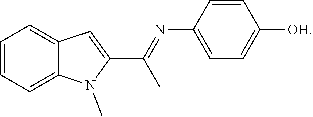 [SMILES: C/C(=N\c1ccc(O)cc1)c1cc2ccccc2n1C]

5. The method of claim 2, wherein the Gαᵢ2 inhibitor is

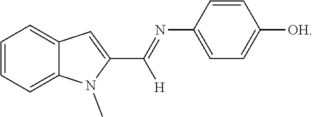 [SMILES: Cn1c(/C=N/c2ccc(O)cc2)cc2ccccc21]

6. The method of claim 2, wherein the Gαᵢ2 inhibitor is

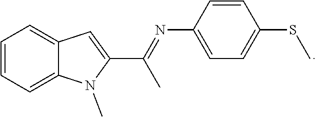 [SMILES: CSc1ccc(/N=C(\C)c2cc3ccccc3n2C)cc1]

7. The method of claim 2, wherein the Gαᵢ2 inhibitor is

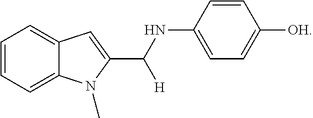 [SMILES: Cn1c(CNc2ccc(O)cc2)cc2ccccc21]

8. The method of claim 2, wherein the Gαᵢ2 inhibitor is

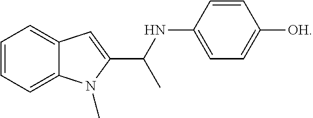 [SMILES: CC(Nc1ccc(O)cc1)c1cc2ccccc2n1C]

9. The method of claim 2, wherein the Gαᵢ2 inhibitor is

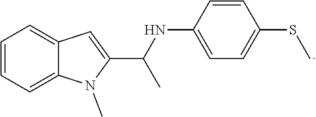 [SMILES: CSc1ccc(NC(C)c2cc3ccccc3n2C)cc1]

10. The method of claim 2, wherein the Gαᵢ2 inhibitor is

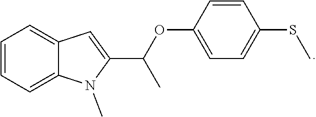 [SMILES: CSc1ccc(OC(C)c2cc3ccccc3n2C)cc1]

11. The method of claim 2, wherein the Gαᵢ2 inhibitor is

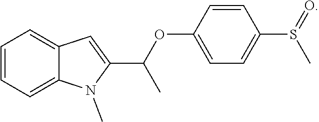 [SMILES: CC(Oc1ccc(S(C)=O)cc1)c1cc2ccccc2n1C]

12. The method of claim 2, wherein the Gαᵢ2 inhibitor is

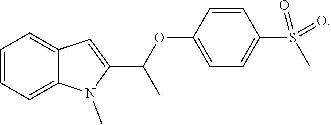 [SMILES: CC(Oc1ccc(S(C)(=O)=O)cc1)c1cc2ccccc2n1C]

13. The method of claim 1, wherein the chemotherapeutic drug induces cell migration if the subject does not receive the Gαᵢ2 inhibitor.

14. The method of claim 1, wherein the Gαᵢ2 inhibitor inhibits cancer cell migration that is induced by the chemotherapeutic drug.

15. The method of claim 1, wherein the cancer is selected from the group consisting of prostate cancer, breast cancer, and ovarian cancer.

16. A method of reducing cell migration in a subject that is receiving a chemotherapeutic drug to inhibit cancer cells, the method comprising administering a Gαᵢ2 inhibitor.

17. The method of claim 16, wherein the Gαᵢ2 inhibitor is a compound of Formula I having the following structure: 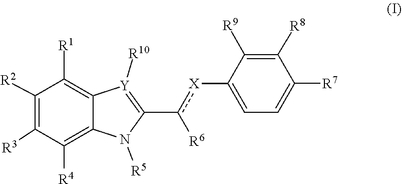
wherein [symbol]
is a single or double bond,
R¹, R², and R³ are each independently H, —OH, —NH₂, or halogen;
R⁴ is H, alkyl, halo-alkyl, aryl, pyridyl, wherein the aryl and pyridyl groups are optionally substituted at the ortho, meta, and para positions;
R⁵ is H, methyl, or ethyl;
R⁶ is H, methyl, or ethyl;
R⁷ is —OH, -OMe, -SMe, -SOMe, or —SO₂Me;
R⁸ and R⁹ are each independently H or —C(O)NH₂;
R¹⁰ is H, —OH, or halogen;
X is N or O; and
Y is C or N.

18. The method of claim 16, wherein the Gαᵢ2 inhibitor is selected from the group consisting of:

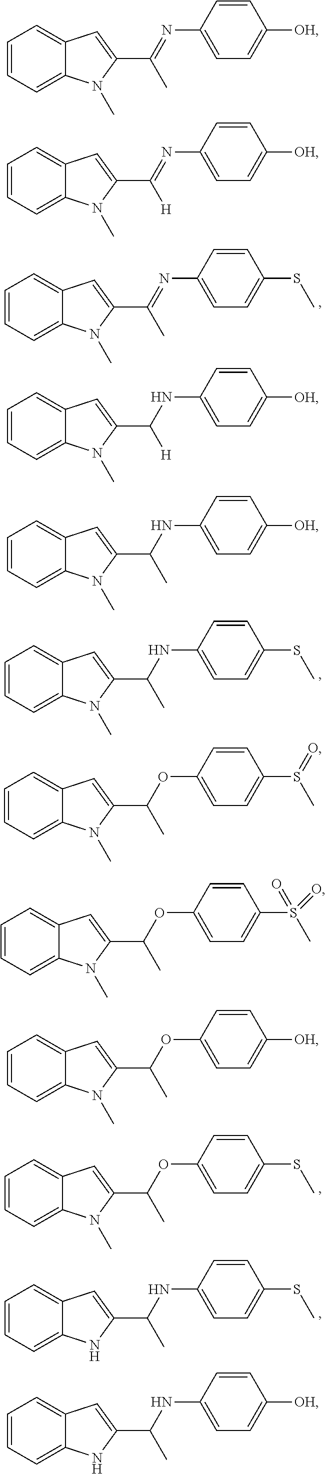

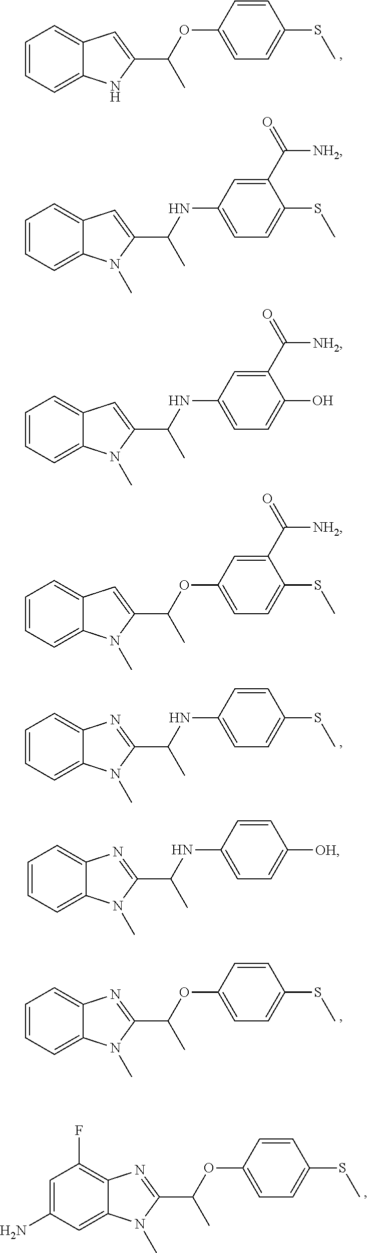

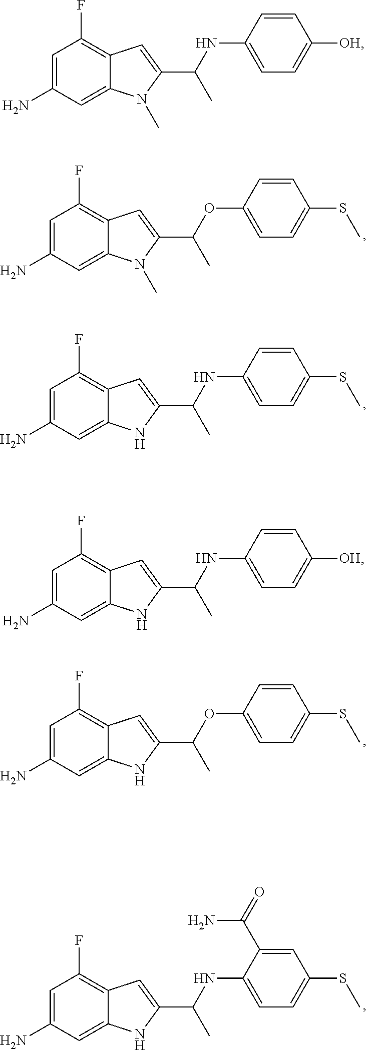 [SMILES: CC(Nc1ccc(O)cc1)c1cc2c(F)cc(N)cc2n1C ; CSc1ccc(OC(C)c2cc3c(F)cc(N)cc3n2C)cc1 ; CSc1ccc(NC(C)c2cc3c(F)cc(N)cc3[nH]2)cc1 ; CC(Nc1ccc(O)cc1)c1cc2c(F)cc(N)cc2[nH]1 ; CSc1ccc(OC(C)c2cc3c(F)cc(N)cc3[nH]2)cc1 ; CSc1ccc(NC(C)c2cc3c(F)cc(N)cc3[nH]2)c(C(N)=O)c1]

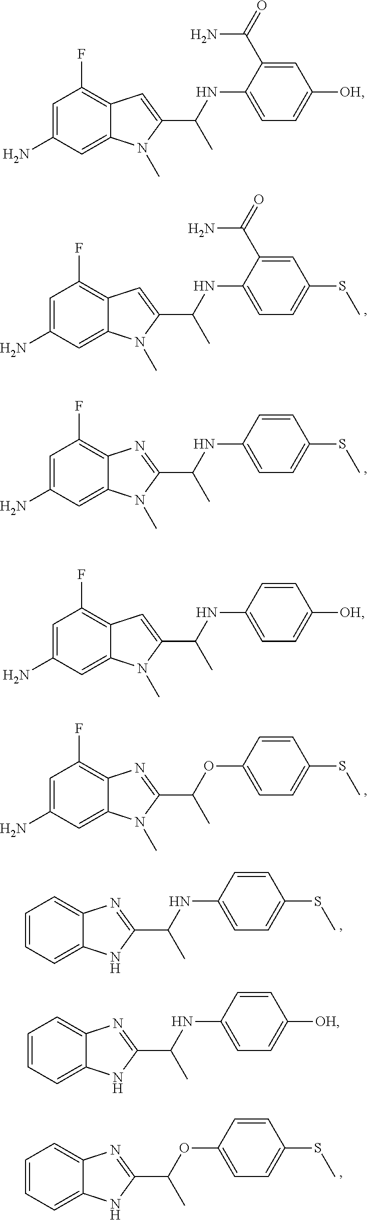

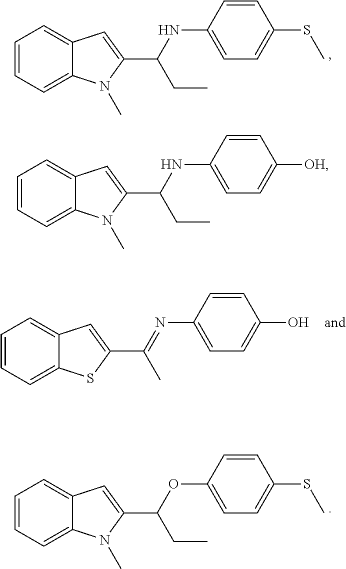 [SMILES: CCC(Nc1ccc(SC)cc1)c1cc2ccccc2n1C ; CCC(Nc1ccc(O)cc1)c1cc2ccccc2n1C ; C/C(=N\c1ccc(O)cc1)c1cc2ccccc2s1 ; "and" ; CCC(Oc1ccc(SC)cc1)c1cc2ccccc2n1C]

19. The method of claim 16, wherein the chemotherapeutic drug induces cell migration if the subject does not receive the Gαᵢ2 inhibitor.

20. The method of claim 16, wherein the Gαᵢ2 inhibitor inhibits cancer cell migration that is induced by the chemotherapeutic drug.
